(19) United States
(12) Patent Application Publication
(10) Pub. No.: US 2013/0018395 A1
(43) Pub. Date: Jan. 17, 2013

(54) SURGICAL IMPLANT DEPLOYMENT DEVICE

(71) Applicants: Jack David Friedlander, Rocklin, CA (US); Dylan M. Hushka, Gilbert, AZ (US); Jeffery D. Arnett, Gilbert, AZ (US)

(72) Inventors: Jack David Friedlander, Rocklin, CA (US); Dylan M. Hushka, Gilbert, AZ (US); Jeffery D. Arnett, Gilbert, AZ (US)

(73) Assignees: Evolap, LLC, Puyallup, WA (US); IMDS Corporation, Logan, UT (US)

(21) Appl. No.: 13/566,901

(22) Filed: Aug. 3, 2012

Related U.S. Application Data
(63) Continuation-in-part of application No. 12/587,458, filed on Oct. 7, 2009.
Application No. 12/587,458 is a continuation-in-part of application No. 61/574,446, filed on Aug. 3, 2011.

(51) Int. Cl. A61B 17/03

(52) U.S. Cl. 606/151

(57) ABSTRACT

An apparatus, method, and system for the deployment of surgical mesh material, which are particularly suited for use in the laparoscopic surgical repair of hernias. Any suitable surgical mesh can be placed between at least two elongate retaining members; wrapped around the elongate retaining members; inserted into a patient; and then deployed using at least two elongate deploying members on either side of the mesh.

### CROSS-REFERENCE TO RELATED APPLICATIONS

This application is a continuation-in-part of:

pending prior U.S. patent application Ser. No. 12/587,458 filed Oct. 7, 2009, which carries Attorney Docket No. 362296.20001, and is entitled: APPARATUS, METHOD AND SYSTEM FOR THE DEPLOYMENT OF SURGICAL MESH.

This application also claims the benefit of:

U.S. Provisional Patent Application No. 61/574,446, filed Aug. 3, 2011, which carries Attorney Docket No. 362296.00100, and is entitled: SURGICAL MESH DEPLOYMENT APPARATUS AND METHOD.

The above-identified documents are incorporated herein by reference in their entirety.

### BACKGROUND

The present disclosure relates to surgical instrumentation, and more particularly to an apparatus, method, and system for the deployment of a surgical mesh inside of a patient. While the present disclosure is made in the context of hernia repair, it is understood that the principles herein may be applicable in other surgical implant deployment applications as well as in non-surgical applications.

Hernias are protrusions of an organ, or the fascia of an organ, through the wall of the cavity that normally contains the organ. By far the most common types of hernias develop in the abdomen, when a weakness in the abdominal wall evolves into a localized hole, or “defect”, through which adipose tissue, or abdominal organs covered with peritoneum, may protrude.

Surgical repair is necessary in order to prevent complications of incarceration (contents become trapped in the defect) and strangulation (blood supply to the contents becomes compromised) which can lead to significant morbidity and potential mortality. Inguinal hernias are defects in the lower abdominal wall and are the most common types of hernias, and thus, repair of inguinal hernias is one of the most commonly performed general surgical procedures. Another type of hernia is ventral, or incisional, hernias which form in the anterior abdominal wall and frequently occur at the site of a previous operative incision, though they may also occur without prior surgery.

Laparoscopic or Minimally Invasive Surgical (MIS) techniques are well-known, widely utilized surgical techniques. Laparoscopic or MIS surgical techniques for hernia repair are preferable to open surgery because MIS surgery uses smaller surgical incisions, which allow the patient to recover faster and reduce the risk of complications during surgery. Laparoscopic surgery is performed by inflating the abdominal cavity with carbon dioxide gas followed by insertion of a number of thin cannulas through the abdominal wall. A video scope is usually placed through one of the cannulas, and long thin operating instruments are placed through other cannulas. The cannulas commonly used in laparoscopic surgery have inner diameters of 5 millimeters (mm), 10 mm, 12 mm, and less commonly 15 mm.

Hernias of the abdominal wall can be repaired using laparoscopic techniques by the placement of surgical reinforcement prosthesis (i.e. a surgical mesh material) inside the abdominal cavity or in the floor of the inguinal canal, and against the hernia defect. This procedure repairs the hernia defect and imparts the aforementioned advantages of laparoscopic surgery to the repair of abdominal wall and inguinal hernias.

Laparoscopic ventral hernia repair is performed in several steps:

(1) Preparing the mesh outside the patient, which includes rolling, folding, or otherwise conforming the mesh to a mesh deployment device such that it may be passed through a surgical cannula into the patient; (2) Inserting the mesh into the patient through a surgical cannula of limited internal diameter without damaging the mesh or causing harm to the patient; (3) Unfurling the mesh in the proper orientation inside the patient, such that the proper surface of the mesh is facing up toward the abdominal wall, and the mesh is properly oriented to the size, shape, and location of the hernia defect in the abdominal wall (This is usually accomplished by grasping and manipulating the mesh with laparoscopic surgical graspers); (4) Once oriented, the mesh is elevated up to the abdominal wall adjacent the hernia defect. This is usually performed using surgical graspers and four-point traction; and (5) once in place, the mesh can be tacked or sutured to the tissue and the surgical implant deployment device can then be removed from the patient.

Numerous difficulties are encountered in laparoscopic hernia repair in the first four phases of the procedure described above. Preparation of the mesh on the outside of the patient may include placement of orienting or fixation sutures onto the edges of, or in the center of the mesh, adding time and complexity to the surgical procedure. Other difficulties are associated with rolling, folding or otherwise conforming the mesh to fit within the inner diameter of the surgical cannula through which it must be passed. The bulk of the mesh/prosthesis material limits the size of the mesh which can be passed through the cannula. The mesh must be conformed, by rolling, folding, or other manipulations such that it can be passed through the cannula to the inside of the patient without causing damage to the mesh or injury to the patient. In some cases, the mesh may be so large that it must be passed through an enlarged skin incision directly instead of through a cannula. This increases the risk of contaminating the mesh with infectious agents. This also increases tissue and/or organ damage, and operating time.

Mesh materials are not inherently rigid and additional rigidity is required to safely pass the mesh through a surgical cannula. In some surgical practices, the mesh is rolled around a laparoscopic surgical instrument in order to impart this rigidity. However, the diameter of the surgical instrument limits the size of the mesh that can be placed through the fixed inner diameter of the surgical cannula.

Another difficulty relates to unfurling and orienting the mesh inside the patient. This process is typically performed by using laparoscopic grasping and suturing instruments. Proper orientation of the mesh includes unfurling the mesh with the correct side of the mesh facing upwards, towards the abdominal wall, so the correct side of the mesh can be placed against the hernia defect. This process can be difficult and time consuming.

Another difficulty involves elevating the mesh up toward the herniated tissue. Typically, the mesh is elevated by passing a long thin suture grasping instrument through the abdominal wall, grasping each of the four fixation sutures, and pulling them up through the abdominal wall. Grasping and manipulating the sutures requires significant laparoscopic surgical skills, which most surgical practitioners lack. It is often difficult to properly orient and tension the mesh using this technique. Accordingly, it would be desirable to have a mesh deployment system including: 1) a small enough diameter to allow the device holding the mesh to fit through the a cannula of limited diameter; 2) an unfurling mechanism that easy to use, and can properly orient and place the mesh; and 3) easy disengagement of the mesh from the deployment device, once the mesh is in place.

Moreover, surgical meshes are typically made to interact with a specific mesh deployment device to load, hold, deploy, and release the mesh. For example, some surgical meshes use pockets, sleeves, or other structures tailored to interact with a specific surgical mesh deployment device to hold, deploy, and release the mesh. It would be desirable to create a surgical mesh deployment device that can work with any style, size, or shape mesh to reduce the number and cost of surgical mesh deployment devices necessary to perform hernia surgeries. This would also give the surgeon the ability to use any style or size mesh he/she desires.

### BRIEF DESCRIPTION OF THE DRAWINGS

Various embodiments of the present disclosure will now be discussed with reference to the appended drawings. It will be appreciated that these drawings depict only typical examples of the present disclosure and are therefore not to be considered limiting of the scope of the invention.

FIG. 1A is an exploded view of a mesh deployment apparatus according to the present disclosure;

FIG. 1B illustrates a surgical mesh for use with the mesh deployment apparatus of FIG. 1A;

FIG. 1C illustrates an alternate embodiment of a surgical mesh for use with the mesh deployment apparatus of FIG. 1A;

FIGS. 2A-2E illustrate the operation of the deployment apparatus of FIG. 1A with the mesh of FIG. 1B;

FIGS. 3A and 3B are side and bottom views of the actuator rod assembly of FIG. 1A;

FIGS. 4A and 4B show an enlarged view of the actuator tip of the deployment apparatus of FIG. 1A;

FIG. 4C shows an enlarged view of the deployment arms engaged with the actuator rod tip;

FIGS. 5A-5D illustrate various embodiments for the actuator rod tip and deployment arm tips;

FIG. 6 shows an enlarged view of the underside of the grip;

FIGS. 7A-7D illustrate the operation of the actuating pin and the notch in the grip.

FIG. 8 is an isometric view of a surgical implant deployment device with a deployed surgical implant on the distal end;

FIG. 9 is a close-up isometric view of the distal end of the surgical implant deployment device of FIG. 1 with the surgical implant removed;

FIG. 10 is an isometric view of the surgical implant deployment device of FIG. 1 in the retracted position with the distal ends split apart;

FIG. 11 is an isometric view of a C-shaped roller;

FIG. 12 is another isometric view of the C-shaped roller of FIG. 4;

FIG. 13 is an isometric view of the C-shaped roller of FIG. 4 attached to a surgical implant deployment device;

FIG. 14 is an isometric view of the surgical implant deployment device of FIG. 1 in the retracted position;

FIG. 15 is an isometric view of the surgical implant deployment device of FIG. 1 in a partially deployed position;

FIG. 16 is an isometric view of the surgical implant deployment device of FIG. 1 in a deployed position;

FIG. 17 is a close-up isometric view of the distal end of the surgical implant deployment device of FIG. 2 with the top tip member removed;

FIG. 18 is a close-up view of the bottom tip member of the surgical implant deployment device shown in FIG. 10;

FIG. 19 is a close-up view of the top tip member of the surgical implant deployment device shown in FIG. 10;

FIG. 20 is an exploded view of the surgical implant women device of FIG. 1;

FIG. 21A is an exploded view of an embodiment of a mesh deployment apparatus according to the present invention;

FIG. 21B is a perspective view of the mesh deployment apparatus of FIG. 21A with mesh mounted on the apparatus;

FIG. 22A is a perspective view of the mesh deployment apparatus of FIG. 21A in a ready configuration;

FIG. 22B is a perspective view of the mesh deployment apparatus of FIG. 21A in a retracted configuration with surgical mesh mounted on the apparatus;

FIG. 22C is a perspective view of the mesh deployment apparatus of FIG. 21A in a loaded configuration;

FIG. 22D is a perspective view of the mesh deployment apparatus of FIG. 21A in a deployed configuration;

FIG. 23A is a perspective view of the of an actuator assembly of the mesh deployment apparatus of FIG. 21A in an open configuration;

FIG. 23B is a perspective view of an actuator assembly of the mesh deployment apparatus of FIG. 21A in a closed configuration;

FIG. 24A is a side view of a latch mechanism of the actuator assembly of FIG. 23B in a closed configuration;

FIG. 24B is a perspective view of a latch release mechanism of the mesh deployment apparatus of FIG. 21A in a closed configuration; and

FIG. 25 is a perspective detailed view of the distal portion of the mesh deployment apparatus of FIG. 21A with the deployment arms in a deployed position and the latch mechanism in its closed configuration.

### DETAILED DESCRIPTION

In this specification, standard medical directional terms are employed with their ordinary and customary meanings. Superior means toward the head. Inferior means away from the head. Anterior means toward the front. Posterior means toward the back. Medial means toward the midline, or plane of bilateral symmetry, of the body. Lateral means away from the midline of the body. Proximal means toward the trunk of the body. Distal means away from the trunk.

In this specification, a standard system of three mutually perpendicular reference planes is employed. A sagittal plane divides a body into bilaterally symmetric right and left portions. A coronal plane divides a body into anterior and posterior portions. A transverse plane divides a body into superior and inferior portions.

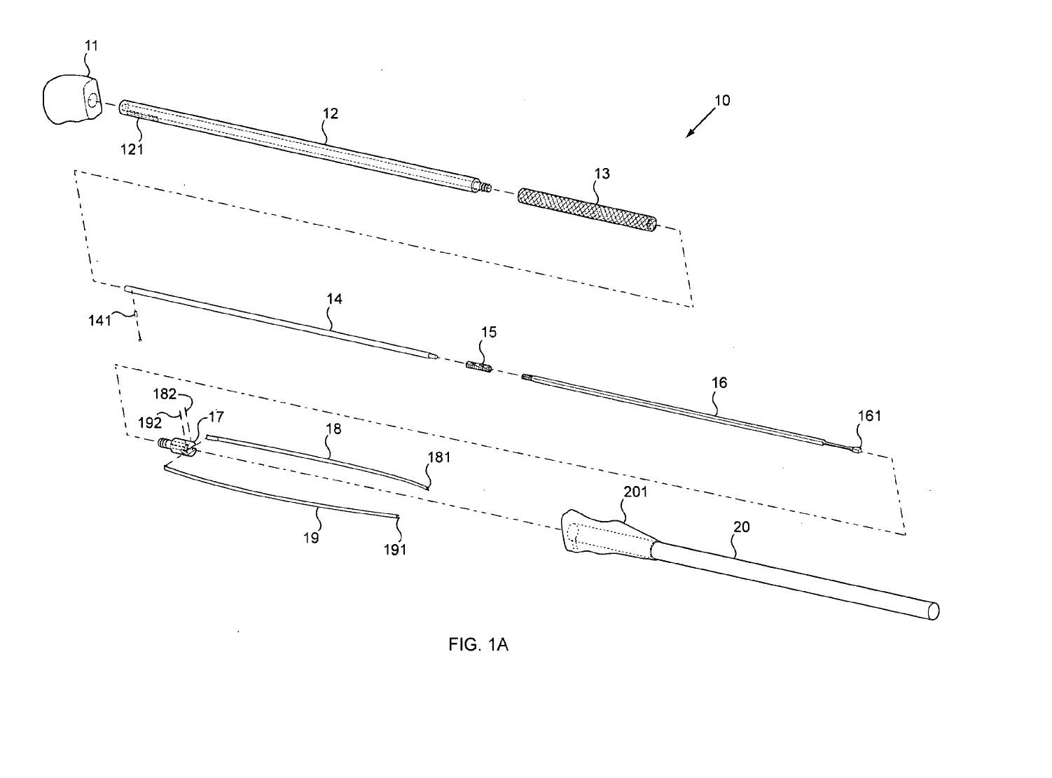

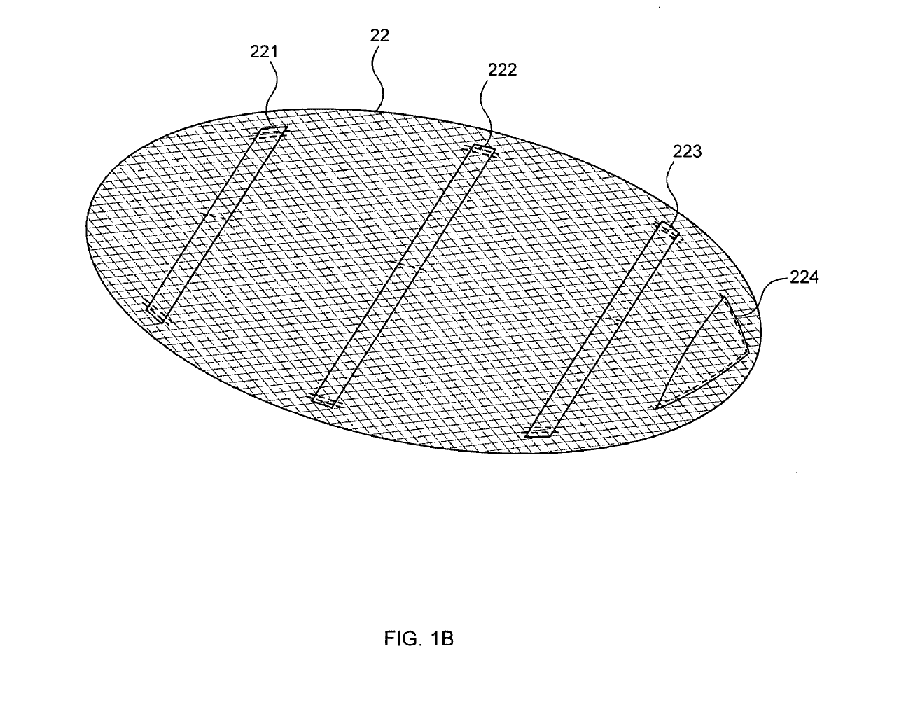

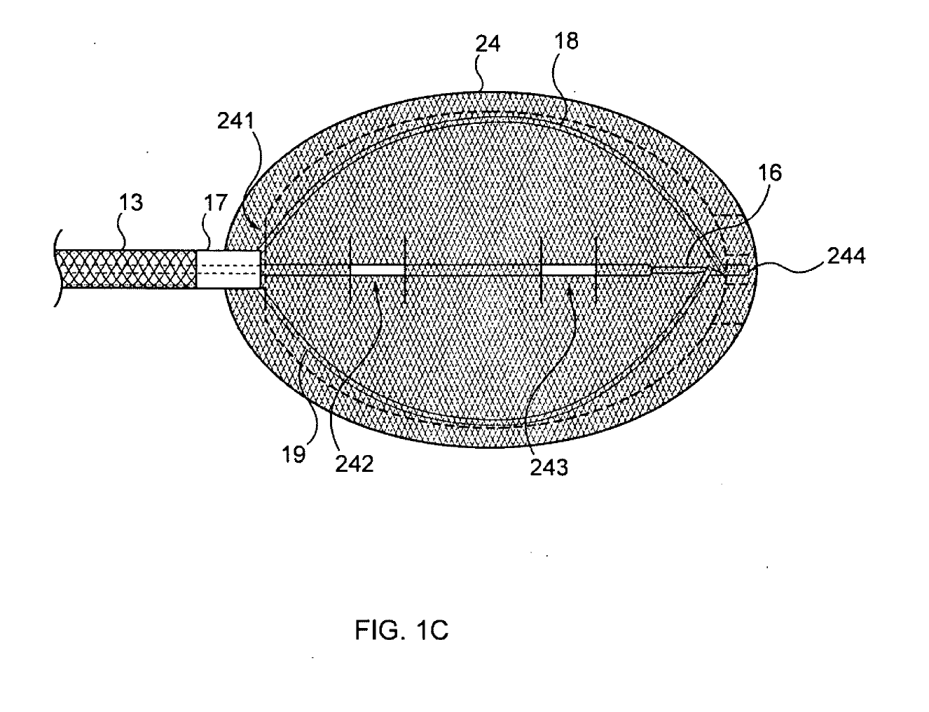

FIG. 1A is an exploded view of a mesh deployment apparatus 10 according to an embodiment of the present disclosure. The apparatus includes a main shaft 12 having a handle 11 affixed to one end. The main shaft 12 is preferably made from metal or biocompatible plastic. The main shaft 12 is hollow and includes a slot 121 near the handle 11, for receiving an actuating pin 141. A hollow flexible tube 13 can be attached to the other end of the main shaft 12. The flexible tube 13 provides flexibility to the apparatus 10 allowing for a greater range of motion to position and manipulate the mesh.

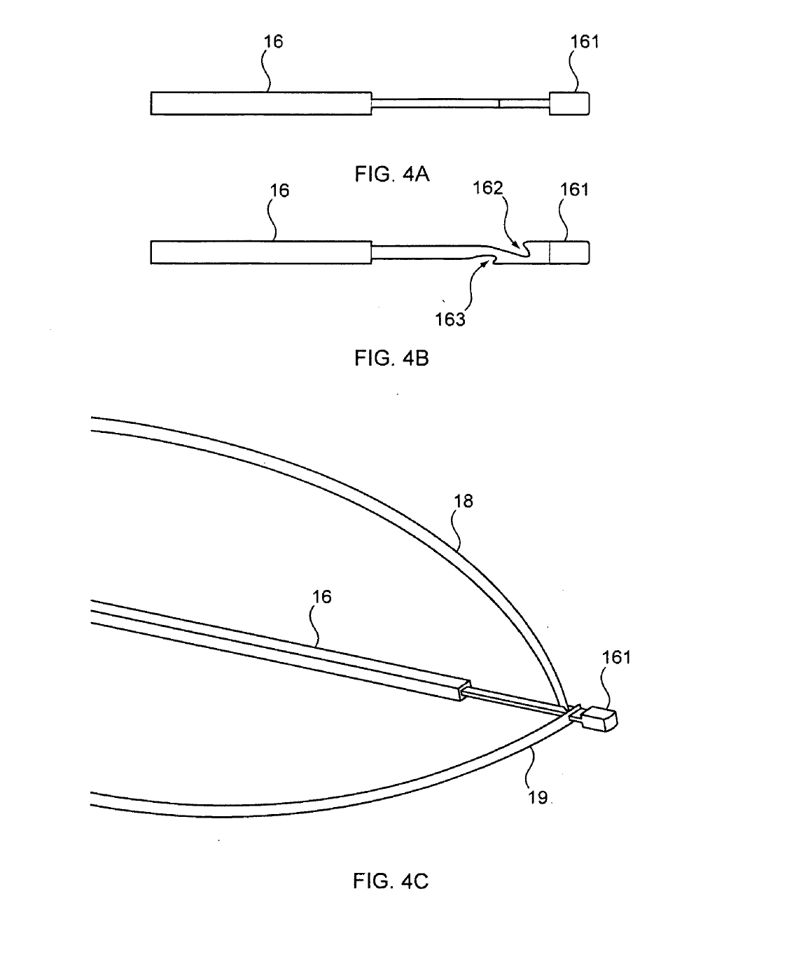

A connecting rod 14 is connected to an actuator rod 16 via a flexible joint 15. The flexible joint 15 is preferably made from a spring or other elastic material. The connecting rod 14 includes an actuating pin 141 that protrudes from slot 121 in the main shaft 12. The flexible joint 15 aligns with the hollow flexible tube 13 when the device is in the deployed position allowing the apparatus 10 to flex for proper positioning of the mesh during use. On the distal end of the actuator rod 16 is a specialized tip 161, shown in detail in FIGS. 4A-4C, and described in detail below. The connecting rod 14, flexible joint 15, and actuator rod 16 are positioned internally to the main shaft 12 and flexible tube 13. Thus, the respective outer diameters of the rods 14, 16 and joint 15 are smaller than the internal diameters of the hollow main shaft 12 and flexible tube 13.

A deployment arm mounting plug 17 can be affixed to an end of the flexible tube 13. The mounting plug 17 includes an internal opening to allow the actuator rod 16 to slide through the mounting plug 17 and is preferably keyed to the actuator rod 16 to prevent rotation about the long axis. Two deployment arms 18, 19 are attached to the mounting plug 17. The deployment arms 18, 19 are preferably formed from spring steel, or similar elastic material which can return to its original shape after flexing. The deployment arms 18, 19 are preferably mounted symmetrically to the plug 17, in the same plane and on opposite sides of the plug 17. The deployment arms 18, 19 are further preferably attached with hinge pins 182, 192, respectively, to allow the deployment arms 18, 19 to freely pivot outward from the mounting plug 17. Each deployment arm 18, 19 includes a notched tip 181, 191, described in greater detail below.

A hollow outer housing 20, including a grip 201, can be positioned to slidingly engage the outer surfaces of the main shaft 12 and flexible tube 13. The outer housing 20 can be manufactured from metal, or any similar rigid material. The mounting plug 17 and deployment arms 18, 19 can slide though the interior of the hollow outer housing 20 in a similar fashion.

Different variations of the above-described embodiment can be utilized. For example, it may not be necessary to have the flexible tube 13 and/or flexible joint 15. Furthermore, different materials may be substituted to construct the various components, as is known in the art.

In a preferred embodiment, the deployment apparatus has an overall length of approximately 30 inches, and the deployment arms 18, 19 are approximately 9 inches long. A preferred surgical mesh is an oval approximately 6×9 inches. However, it will be understood that other dimensions and shapes are within the scope of the present disclosure.

FIG. 1B illustrates a surgical mesh 22 according to an embodiment of the present disclosure, specifically configured to be inter-operable with the deployment apparatus 10 of FIG. 1A. The mesh 22 is preferably made of standard surgical mesh material, such as polypropylene, e-PTFE or other biocompatible materials, and may be coated with any number of adhesion minimizing coatings. The mesh 22 can have an oval shape. Guide loops 221, 222, 223 can be attached to mesh 22 to receive the deployment arms 18, 19. The mesh preferably contains a pouch 224 at one end for inserting the tip of the actuator rod 161. The guide loops 221, 222, 223 may be sewn or otherwise affixed to the surface of the mesh 22. Attachment points can be placed along the centerline of the mesh, to prevent rotation of the mesh, and along the periphery of the mesh to allow the mesh to completely unfurl.

It will be understood that other mesh configurations can be utilized in the present disclosure. For example, FIG. 1C shows mesh 24 which is formed from two oval mesh sheets attached at the outer edges to form an oval pocket with an opening 241 on one end configured to receive deployment arms 18, 19 and actuator rod 16. Center guide loop(s) 242, 243 may be attached to, or formed in the center of the mesh 24, and configured to guide the actuator rod 16. In this embodiment, guide loops may not be required for deployment arms 18, 19. This embodiment can also include a pocket 244 for the actuator rod tip 161.

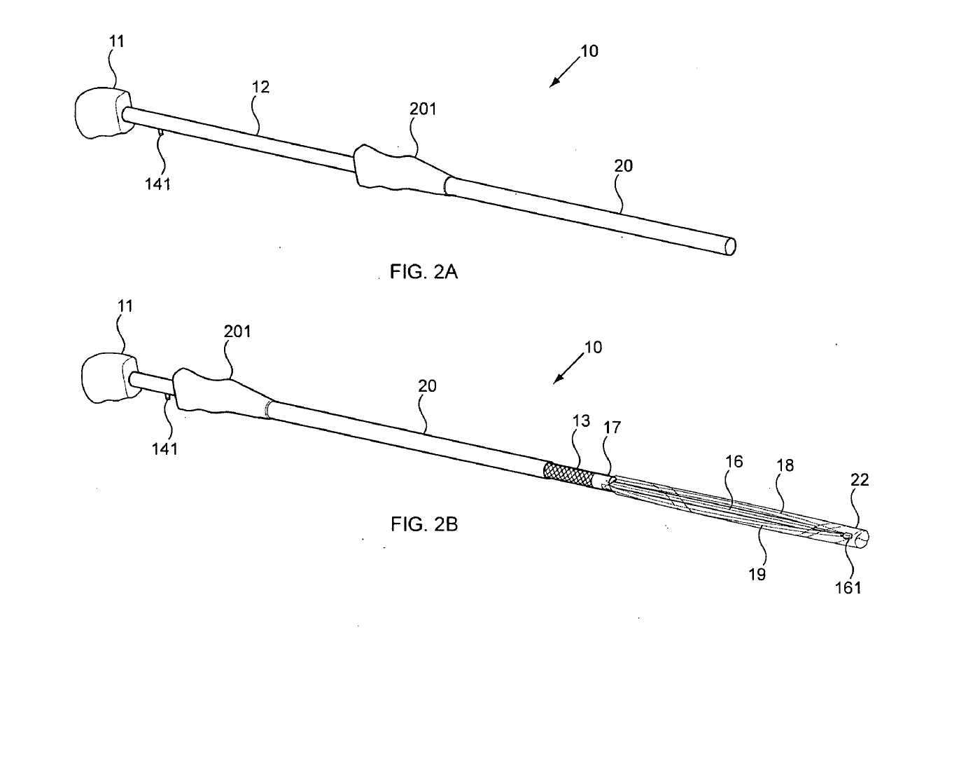

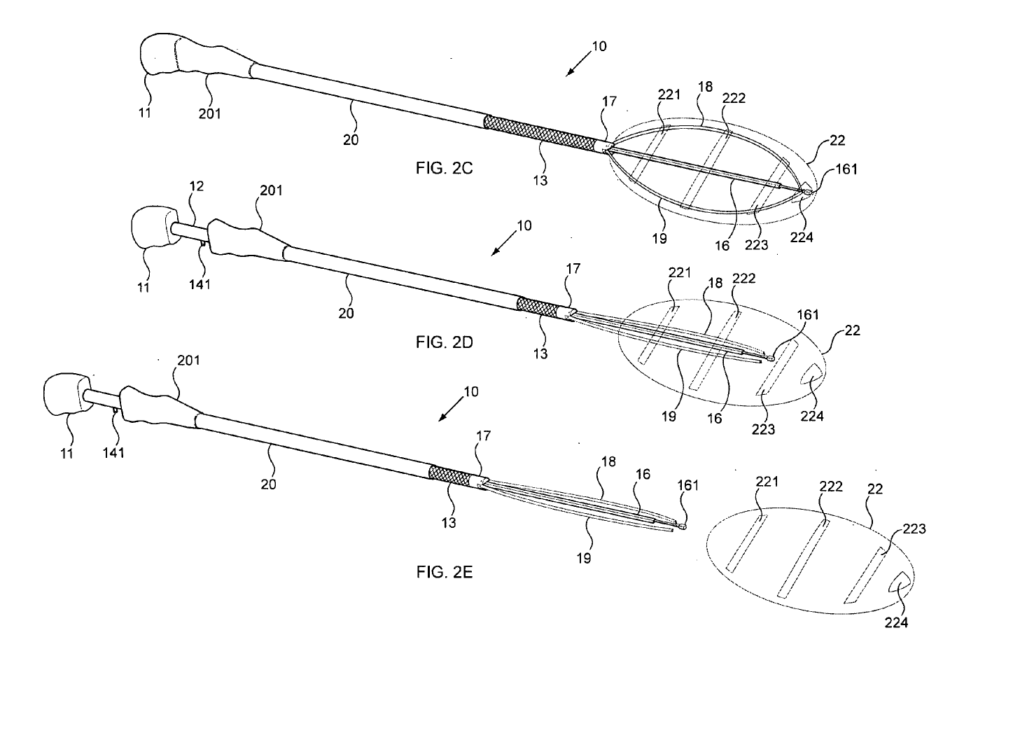

The operation of the fully assembled deployment apparatus 10 is illustrated in FIGS. 2A-2E. As shown in FIG. 2A, the outer housing 20 is slid out to cover the actuator rod 16, deployment arms 18, 19 and mesh 22. In this position, the deployment apparatus 10 can easily be inserted into and positioned through a surgical cannula. Once the outer housing 20 of the deployment apparatus 10 is positioned as desired, the surgeon can push the handle 11 inward, while holding the grip 201 on the outer housing 20. The mesh 22 is exposed as the handle 11 is pushed inward, as illustrated in FIG. 2B. A notch 202 (described in greater detail below) in the end of the grip 201 engages the actuating pin 141, on the connecting rod 14. Once engaged, further inward motion of the handle 11 causes the main shaft 12 with attached flexible tubing 13 and mounting plug 17 to slide over the connecting rod 14, flexible joint 15, and actuator rod 16, while the actuating pin 141 is allowed to slide within the slot 121 in the main shaft 12. As the main shaft 12 is slid over the connecting rod 16, flexible joint 15, and actuator rod 16 the deployment arm tips 181, 191, engaged with the specialized tip 161 of the actuator rod 16, are actively flexed and bowed outward, as shown in FIG. 2C. As the deployment arms 18, 19 bow outward, the surgical mesh 22 is unfurled until it is generally flat and parallel to the surface to which it is to be adhered. The deployment arms 18, 19 can extend outward to the edge of the guide loops to completely flatten the mesh 22.

Once the mesh 22 has been unfurled, positioned, and affixed to the body structure, the handle 11 can be pulled outward. This will release the force on the mounting plug 17 and deployment arms 18, 19, causing the deployment arms 18, 19 to return to their neutral configuration. The actuating pin 141 engaged with slot 202 in the grip 201 fixes the connecting rod 14, flexible joint 15, and actuator rod 16 to the grip 201 allowing the main shaft 12, flexible tubing 13, mounting plug 17, and the deployment arms 18, 19 to move outward relative to the actuator rod 16. This allows the deployment arm tips 181, 191 to fully disengage from the actuator rod tip 161, as illustrated in FIGS. 2D and 2E. Further outward motion of the handle 11 disengages the actuating pin 141 from the notch 202 and allows the actuator rod 16 and deployment arms 18, 19 to be drawn back into the housing 20 for safe removal of the apparatus from the patient.

The mesh 22 can be initially mounted on the actuator rod 16 and deployment arms 18, 19 in the following manner. The actuating pin 141 can slide forward to extend the actuator rod 16 to its maximum extended position. The deployment arms 18, 19 are inserted between the main mesh surface and guide loops 221, 222, 223. Once the deployment arm tips 181, 191 are passed through the last guide loop 223, the deployment arm tips 181, 191 can be engaged with the actuator rod tip 161. Next, the actuator rod tip 161 can be inserted into a mesh pouch 224 at an end of the mesh 22. In this configuration, the deployment arms 18, 19 are under some tension, but are still generally parallel to the actuator rod 16. Finally, the mesh 22 is rolled around, folded around, or otherwise conformed around the deployment arms 18, 19 and actuator rod 16 (as illustrated in FIG. 2B). The outer housing 20 can be slid over the assembly to fully encapsulate the mesh 22, as illustrated in FIG. 2A, and the system is now ready for deployment.

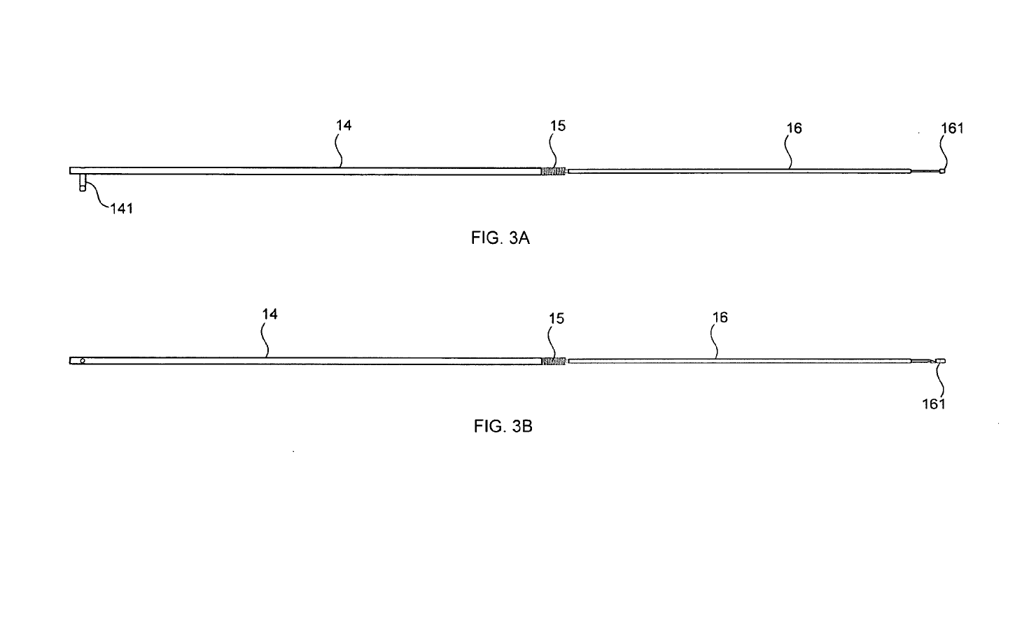

FIG. 3A illustrates the connecting rod 14, flexible joint 15, and actuator rod 16 in side profile. Not that the actuating pin 141 attaches to the connecting rod 14 to slide the entire unit through the main shaft 12, flexible tube 13 and mounting plug 17. FIG. 3B is a bottom view of FIG. 3A.

FIGS. 4A and 4B are enlarged views of the actuator rod tip 161. The actuator rod tip 161 can have notches 162, 163 on each side configured to receive deployment arm tips 181, 191. Each deployment arm 181, 191 can be U-shaped tip and engage a notch on the actuator rod tip 161. FIG. 4C is an enlarged view of the deployment arms 18, 19 engaged with the actuator rod tip 161.

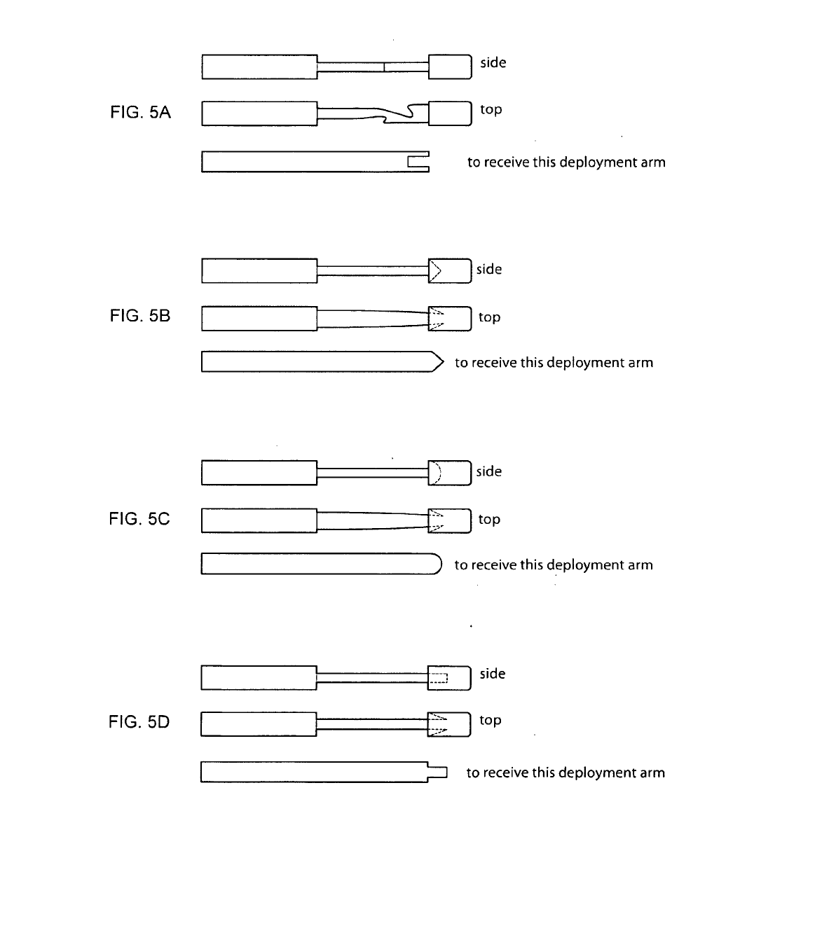

FIGS. 5B-5D illustrate other deployment arm tips embodiments. The specific design of the deployment arm tips is not critical, so long as the tips allow for easy and secure installation and deployment of the mesh 22. Thus, different configurations that allow the deployment arm tips 181, 191 to securely engage with, and fully disengage from the actuator tip 161 are within the scope of the present disclosure. For example, in FIGS. 5B-5D, the actuator tip 161 is formed as a cap having two slots. The slots can be configured to engage with deployment arm tips 181, 191 configured as pointed tips, rounded tips or square tips.

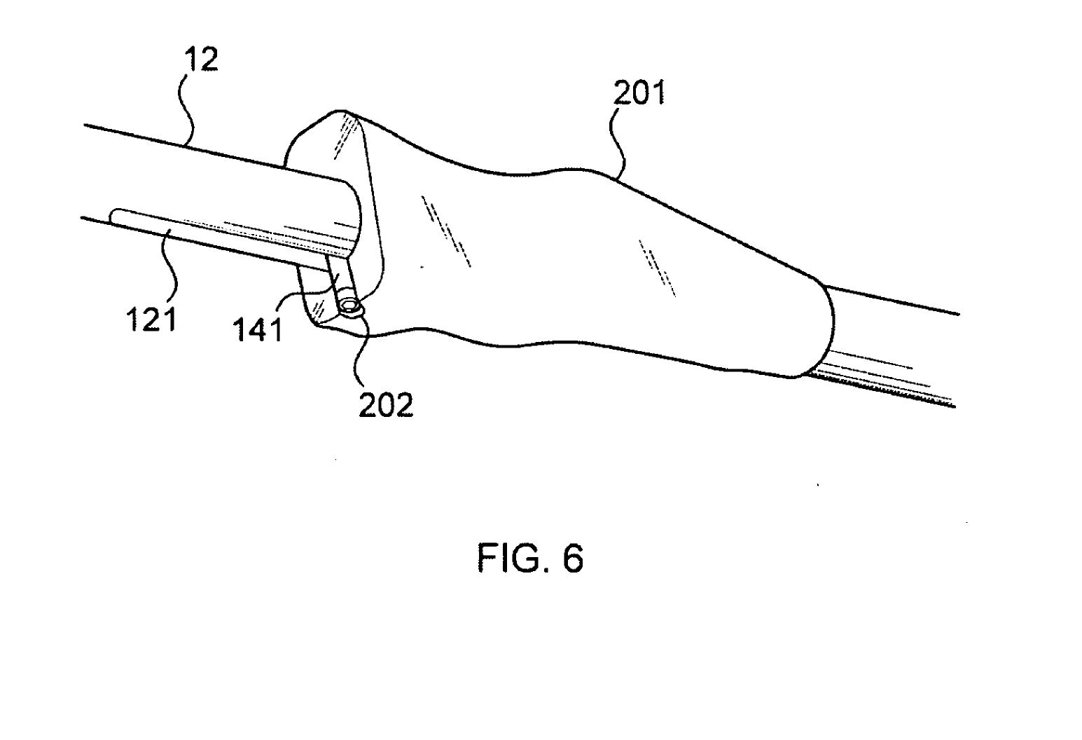

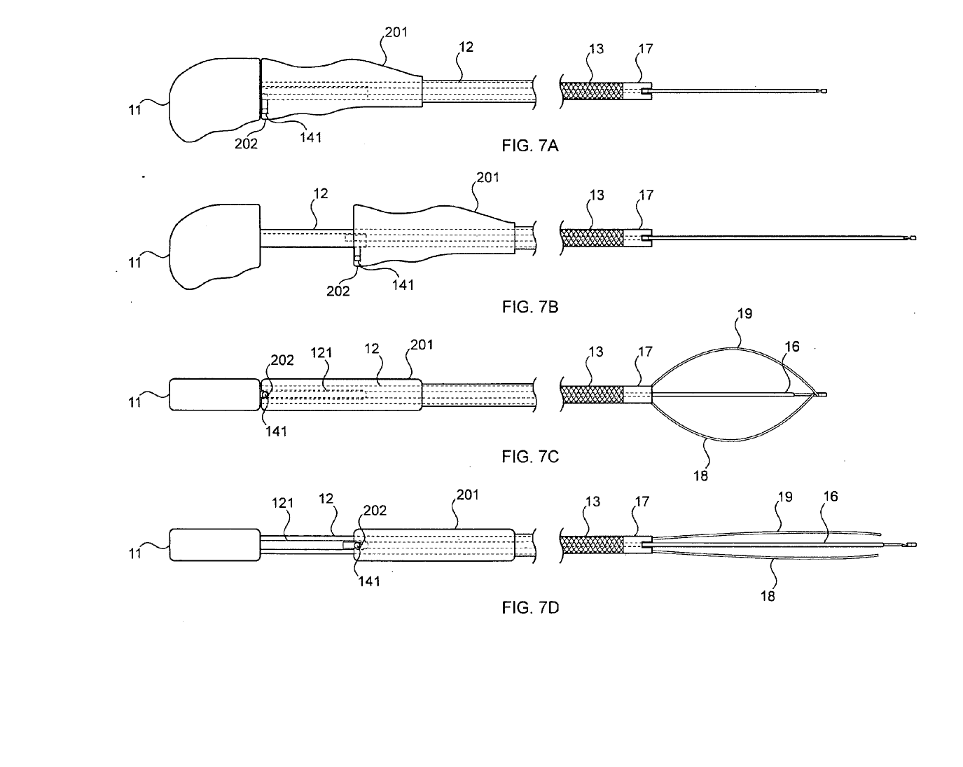

FIG. 6 illustrates the location of the notch 202 in the end of the grip 201, for engaging the actuating pin 141. FIGS. 7A-7D illustrate the operation and engagement of the notch 202 and actuating pin 141 during deployment. FIGS. 7A and 7C illustrate the deployment apparatus 10 with the actuator rod 16 fully retracted. Note that the actuating pin 141 is in the notch 202 of the grip 201. Retracting the actuator rod 16 causes the deployment arms 18, 19 to expand outward. In FIGS. 7B and 7D, the handle 11 is retracted, and the actuating pin 141 is engaged with the notch 202 in the grip 201 (FIG. 7B). This causes the actuator rod 16 to extend, thereby releasing the tips 181, 191 of the deployment arms 18, 19 from the actuator rod tip 161

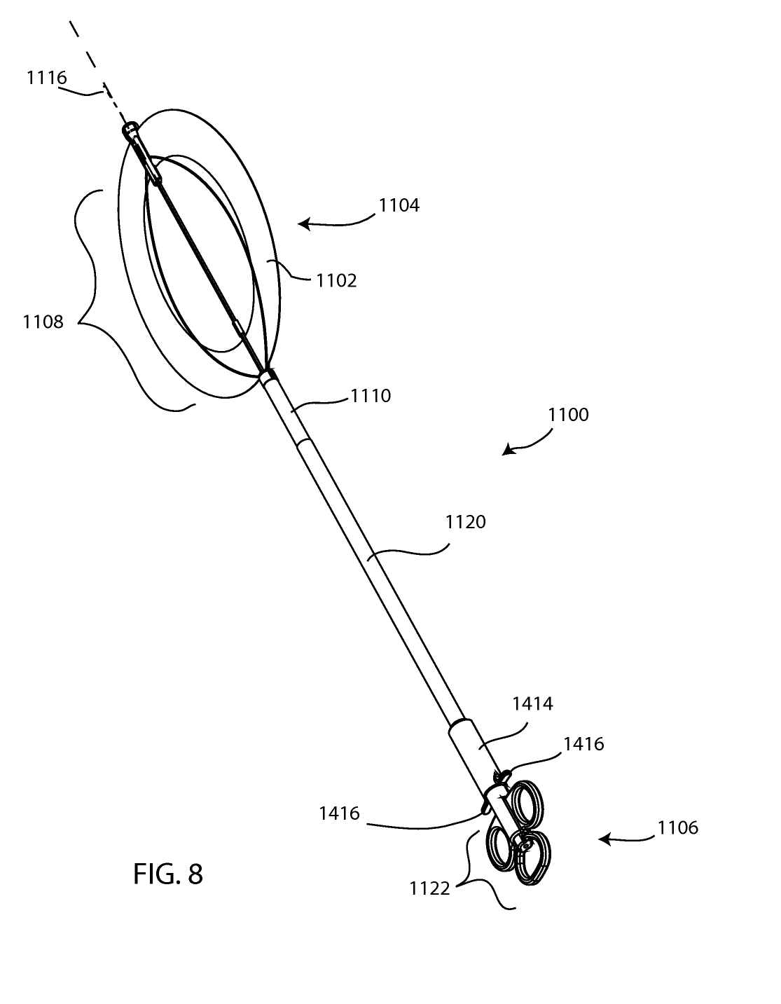

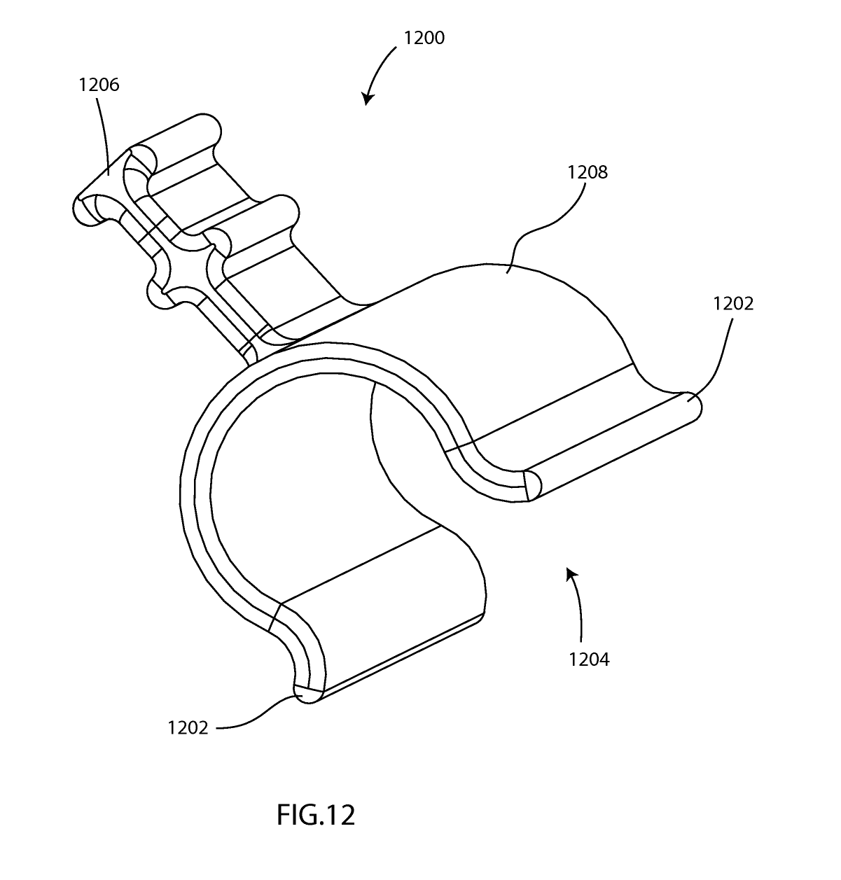

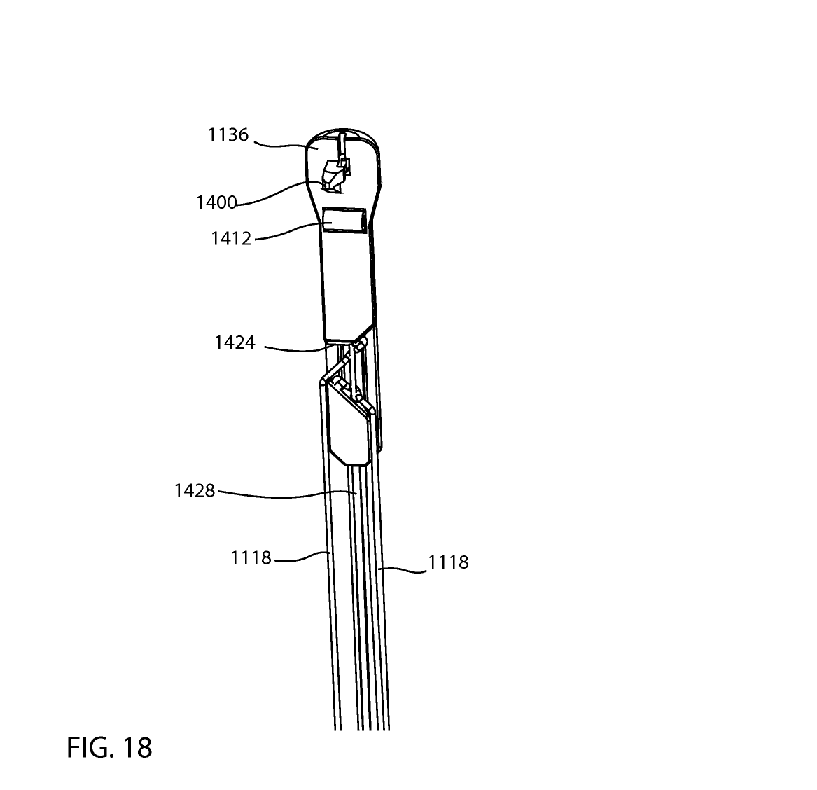

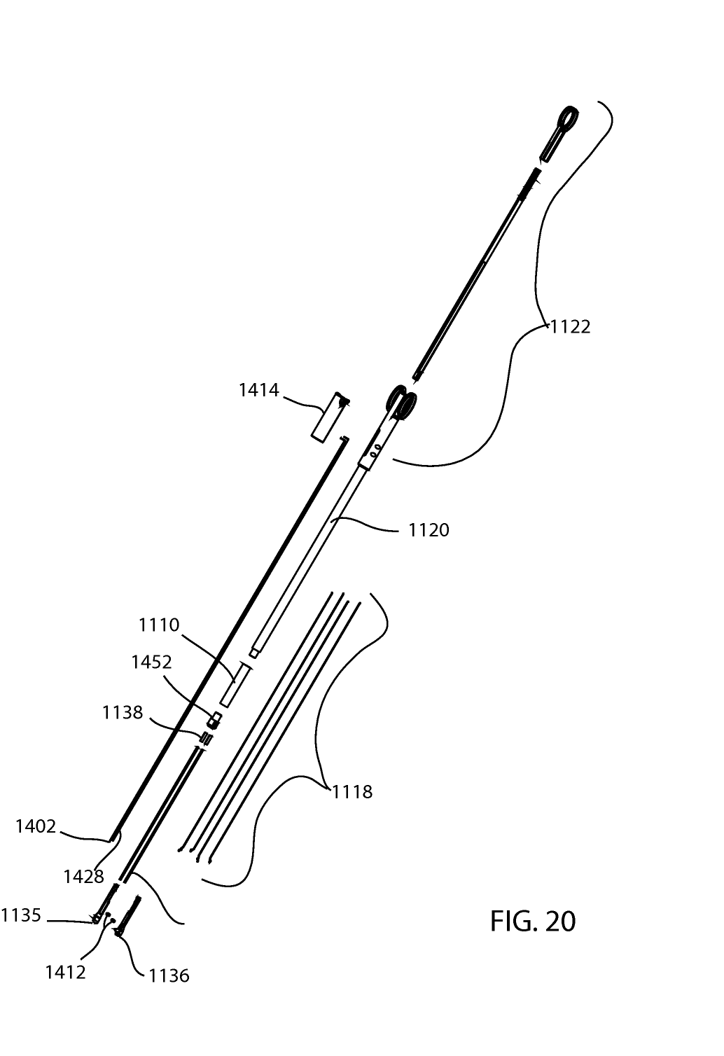

FIGS. 8-20 illustrate a surgical implant deployment device 1100 according to another embodiment of the present disclosure. The surgical implant deployment device 1100 in FIG. 8 facilitates laparoscopic hernia surgery by allowing the surgical implant or mesh 1102 to be “rolled up” and inserted into the patient through a laparoscopic cannula. Once the surgical mesh 1102 is inserted into the patient, the surgeon can unfurl the surgical mesh 1102; position the surgical mesh 1102 next to the herniated tissue; attach the surgical mesh 1102 to the herniated tissue; and then remove the surgical implant deployment device 1100 from the patient.

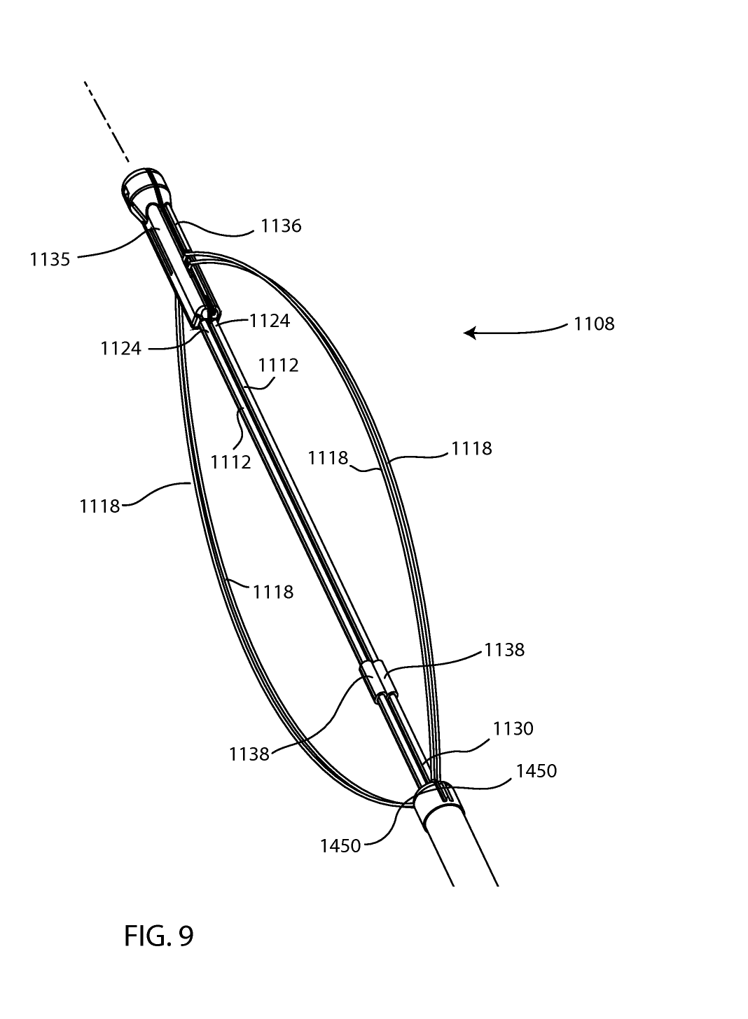

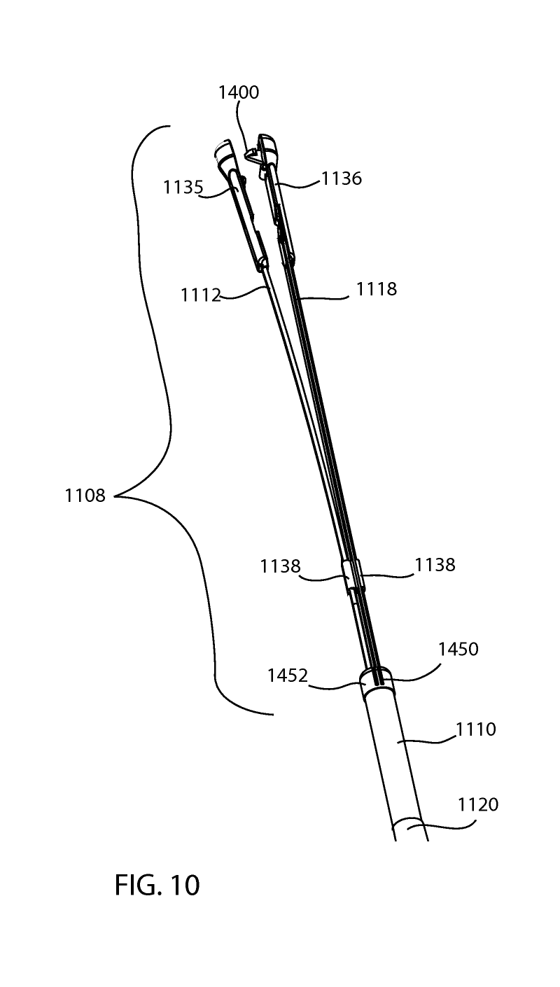

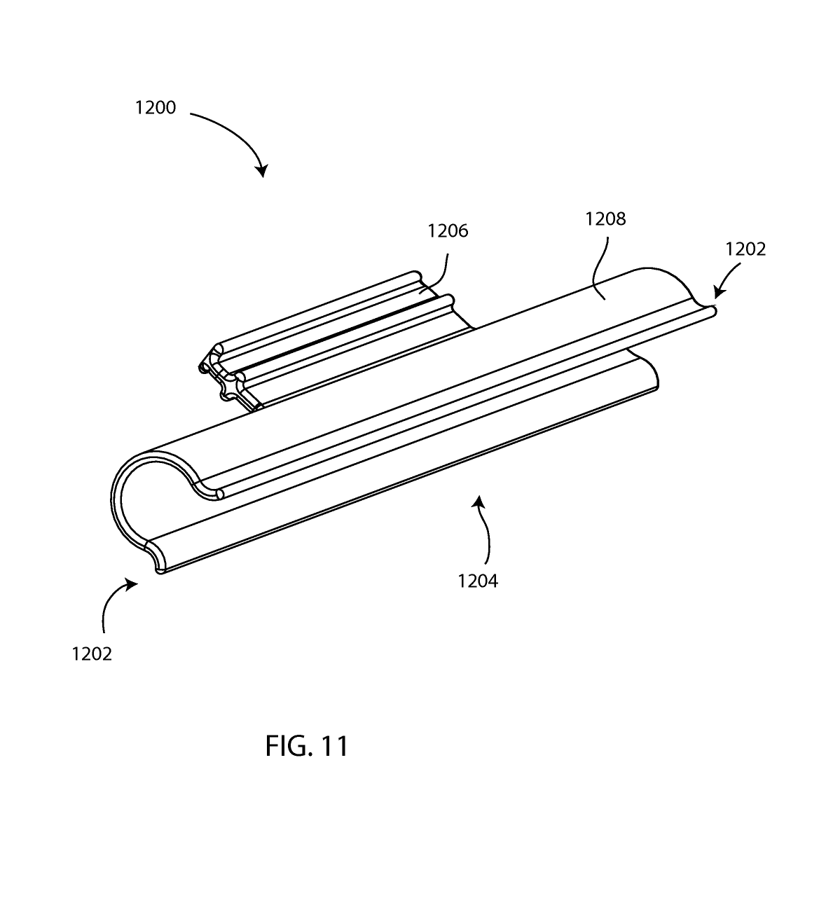

The surgical implant 1102 mounted to the surgical implant deployment device 1100 in FIG. 8 is shown in the “unfurled” or “deployed” position. The surgical implant deployment device 1100 can work with any size, shape, or type of surgical mesh 1102 because the surgical mesh 1102 is simply “sandwiched” between the two halves of the surgical implant deployment member 1108, as will be discussed in more detail with reference to FIGS. 9 and 10. In other words, the deployment device 1100 does not require that the surgical mesh 1102 have pockets, sleeves, sutures, fixation members, or other special structures in order to grip, hold, or deploy the surgical mesh 1102.

Continuing with FIG. 8, the proximal end 1106 of the surgical implant deployment device 1100 can have a deployment actuation member 1122 and a release handle 1414. The deployment device 1100 can have an elongate body 1120 with the surgical implant deployment member 1108 disposed at the distal end 1104 of the elongate body 1120. In some examples, the surgical implant deployment device 1100 can also have a flexible joint 1110 intermediate the surgical implant deployment member 1108 and the elongate body 1120.

FIG. 9 shows a close-up view of the surgical implant deployment member 1108 in the deployed position, without a surgical implant 1102 sandwiched between the two halves of the surgical implant deployment member 1108. The two halves of the surgical implant deployment member 1108 are attached to each other in FIG. 9. In contrast, FIG. 10 shows the surgical implant deployment member 1108 in the retracted or “non-deployed” position, with the distal ends of the two halves of the surgical implant deployment member 1108 released from each other, or “split apart” from each other. In this configuration, any suitable surgical mesh 1102 can be inserted between the two halves of the surgical implant deployment member 1108 to “load” the deployment device 1100 with a surgical implant 1102, or release the surgical implant 1102 from the deployment device 1100 once it has been “tacked” in place. Once the surgical implant 1102 is loaded between the two halves of the surgical implant deployment member 1108, the distal ends of the two halves of the surgical implant deployment member 1108 can be re-attached to each other to “sandwich” the surgical mesh 1102 between the two halves of the surgical implant deployment member 1108 and trap the surgical mesh 1102.

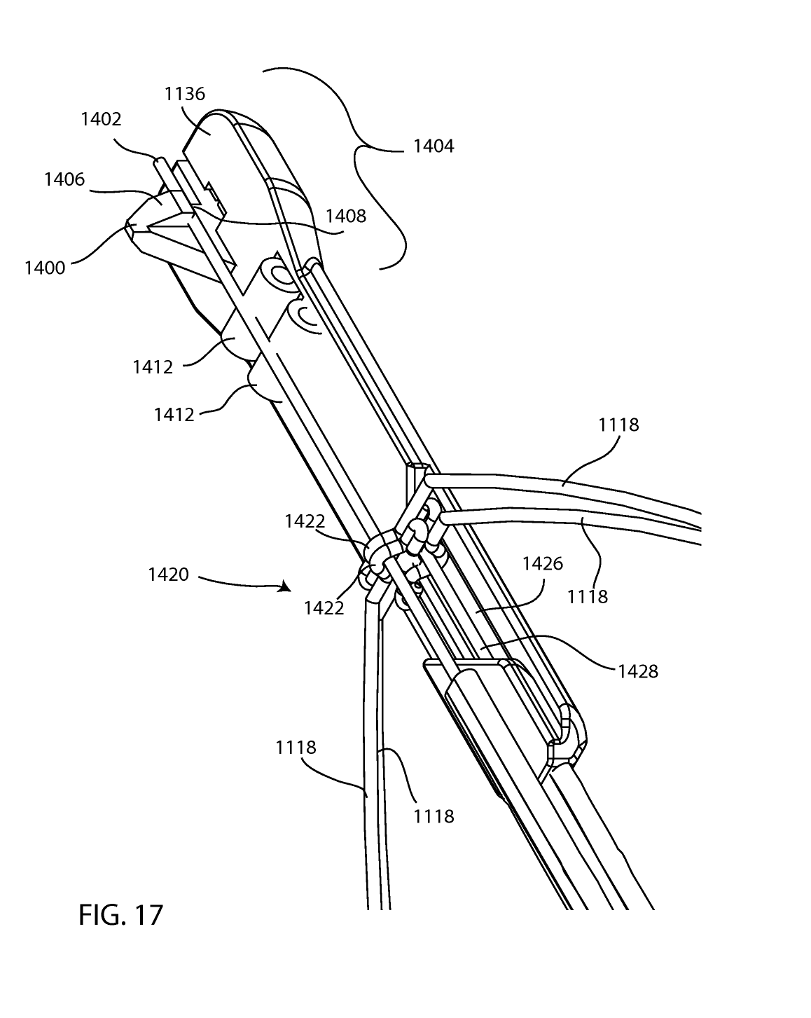

Continuing with FIG. 9, the surgical implant deployment member 1108 can have two or more elongate retaining members 1112 and two or more elongate deploying members 1118. The elongate retaining members 1112 can be arranged opposite each other along the longitudinal axis 1116 of the surgical implant deployment member 1108 with their proximal ends 1130 adjacent the elongated body 1120 and their distal ends 1124 distal from the elongate body 1120. The elongate retaining members 1112 can be rigid, semi rigid, or flexible, and can made of a suitable material such as metal or plastic. The elongate deploying members 1118 can be flexible and have shape memory properties. The elongate deploying members 1118 can be made of a suitable material such as metal or plastic. In examples, the elongate deploying members 1118 are made of nitinol and/or stainless steel. The surgical implant deployment member 1108 can have two or more tip members 1135, 1136 attached to the distal ends of the elongate retaining members 1112. The tip members 1135, 1136 can be releasably attached to each other through an attachment mechanism. The distal ends of the elongate deploying members 1118 can also be releasably attached to the tip members 1135 and 1136, as will be explained in detail more below with reference to FIGS. 17-19.

In other examples, the surgical implant deployment member 1108 may not have tip members 1135, 1136 or an attachment mechanism 1404. For example, the elongate retaining members 1112 can be configured to impart a compressive force between each other without the need of releasably attachable tip members 1135, 1136. Rather, the compressive force can be caused by the shape memory properties inherent in the elongate retaining members 1112 or by a compression member (not shown) configured to impart a compressive force between the elongate retaining members 1112. The deployment device 1100 can also have a compression release member (not shown) configured to decompress the compressive force between the elongate members 1112 to allow for insertion or release of a surgical implant 1102.

The surgical implant deployment member 1108 in FIG. 9 has two pairs of elongate deploying members 1118 on opposite sides of the elongate retaining members 1112. However, in other examples, the surgical implant deployment member 1108 can have one pair of elongate deploying members 1118 along a side of the retaining members 1112, or more than two pairs of elongate deploying members 1118. The top elongate deploying members 1118 of each pair can be releasably attached to the top tip member 1135, and the bottom elongate deploying members 1118 of each pair can be releasably attached to the bottom tip member 1136. In this manner, when the tip members 1135, 1136 are separated from each other, the surgical implant deployment member 1108 can be spread apart into two halves to allow a surgical mesh 1102 to be inserted between the two halves. The first half can include the top tip member 1135, the top elongate retaining member 1112, and the top elongate deploying members 1118. The second half can include the bottom tip member 1136, the bottom elongate retaining member 1112, and the bottom elongate deploying members 1118.

The surgical implant deployment member 1108 can also have one or more grip members 1138 associated with the elongate retaining members 1112. The grip members 1138 can be configured to grip and hold a surgical implant 1102 disposed between the elongate retaining members 1112. In one example, the surgical implant deployment member 1108 has two grip members. In another example, the surgical implant deployment member 1108 has four grip members. In yet another example, the length of the one or more grip members 1138 is adjustable. The grip members 1138 can be made of any suitable material, including but not limited to: polyethylene foam, silicone, plastic, or metal. The grip members 1138 can also be indentations formed in the elongate retaining members 1112 and configured to grip a surgical implant 1102 disposed between the elongate retaining members 1112.

Referring to FIG. 10, the deployment device 1100 can have a flexible joint 1110. The flexible joint can bend to allow the surgeon to orient the surgical implant deployment member 1108. For example, the surgeon can bend the flexible joint 1110 and force the surgical implant deployment member 1108 up toward the herniated tissue to facilitate placement of the surgical implant 1102. The flexible joint 1110 can be made of flexible plastic, surgical tubing, or any other suitable material.

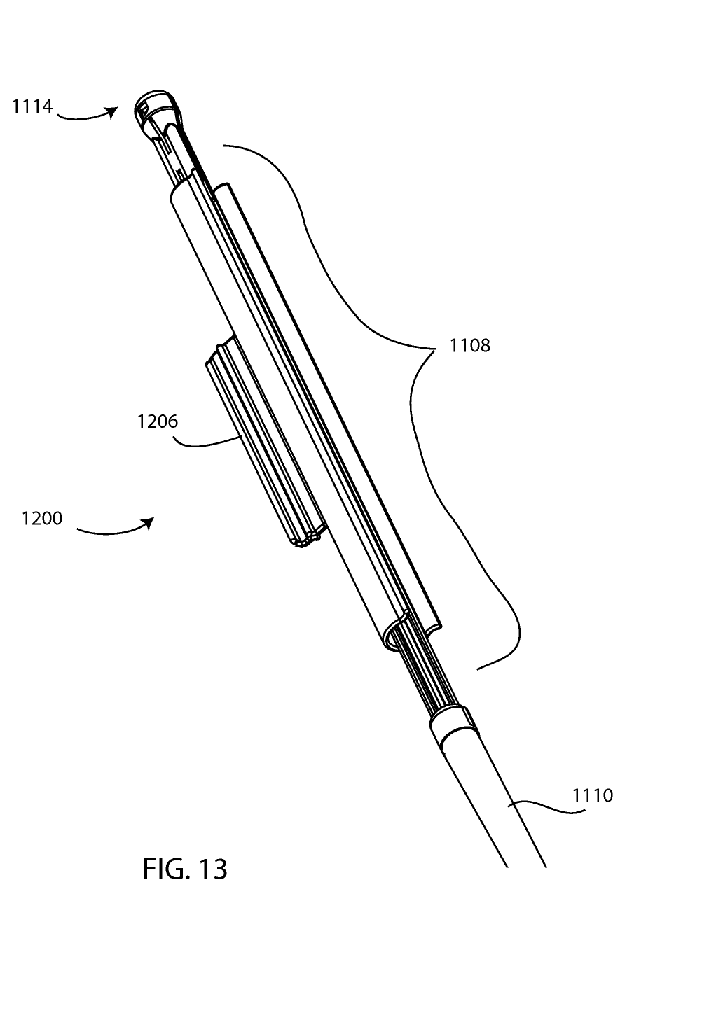

Once the surgical mesh 1102 is sandwiched between the two halves of the surgical implant deployment member 1108 and the tip members 1135, 1136 are attached to each other, the surgical mesh 1102 can be wrapped around the retracted surgical implant deployment member 1108. FIGS. 11-13 show a C-shaped roller 1200 that can be used to wrap the surgical mesh 1102 around the surgical implant deployment member 1108. The C-shaped roller 1200 can also maintain the surgical mesh 1102 in the wrapped configuration prior to insertion into the patient. The C-shaped roller 1200 can have an elongate hollow body 1208 with an opening 1204 along one side of the elongate hollow body 1208 sized to receive the surgical implant deployment member 1108 (in the retracted or “non-deployed” configuration) with a surgical implant 1102. The opening 1204 can have flares 1202 which diverge away from the elongate hollow body 1208 and help guide the retracted surgical implant deployment member 1108 and surgical mesh 1102 into the elongate hollow body 1208, as can be seen in FIG. 13 (surgical mesh 1102 not shown). Once the retracted surgical implant deployment member 1108 and surgical mesh 1102 are inserted into the elongate hollow body 1208, the surgeon can rotate the C-shaped roller 1200 around the surgical implant deployment member 1108 to “wrap” the surgical mesh 1102 around the surgical implant deployment member 1108. The C-shaped roller 1200 can have one or more tabs 1206, or other structures, to facilitate rotation by providing a surface against which the surgeon can apply rotational forces.

Continuing with FIG. 13, the C-shaped roller 1200 can stay attached to the surgical implant deployment member 1108 to hold and maintain the surgical implant 1102 prior to insertion into the patient. The diameter of the elongate hollow body 1208 can be greater than the diameter of the cannula or trocar (not shown) that receives the deployment device 1100 into the patient. This will cause the C-shaped roller 1200 to be pushed proximally along the elongate body 1120 of the deployment device 1100 as the deployment device 1100 is inserted through the cannula and into the patient. Thus, the surgical mesh 1102 will be held in place by the C-shaped roller as it is inserted into the cannula, and the C-shaped roller 1200 will automatically slide backwards and disengage the surgical implant deployment member 1108 upon insertion. The C-shaped roller may then be removed from the deployment device 1100.

The distal ends of the elongate retaining members 1112 or the distal ends of the tip members 1135, 1136 can form an implant shield 1114. The implant shield 1114 can have a diameter that is less than the inner diameter of the cannula (not shown) that receives the deployment device 1100 into the patient, but greater than the diameter of a surgical implant 1102 wrapped around the surgical implant deployment member 1108. The implant shield 1114 can protect the surgical implant 1102 as it is being inserted into the cannula by preventing the surgical implant 1102 from snagging the end or sides of the cannula as it is being inserted. In some examples, the diameter of the implant shield 1114 can be about 12 mm.

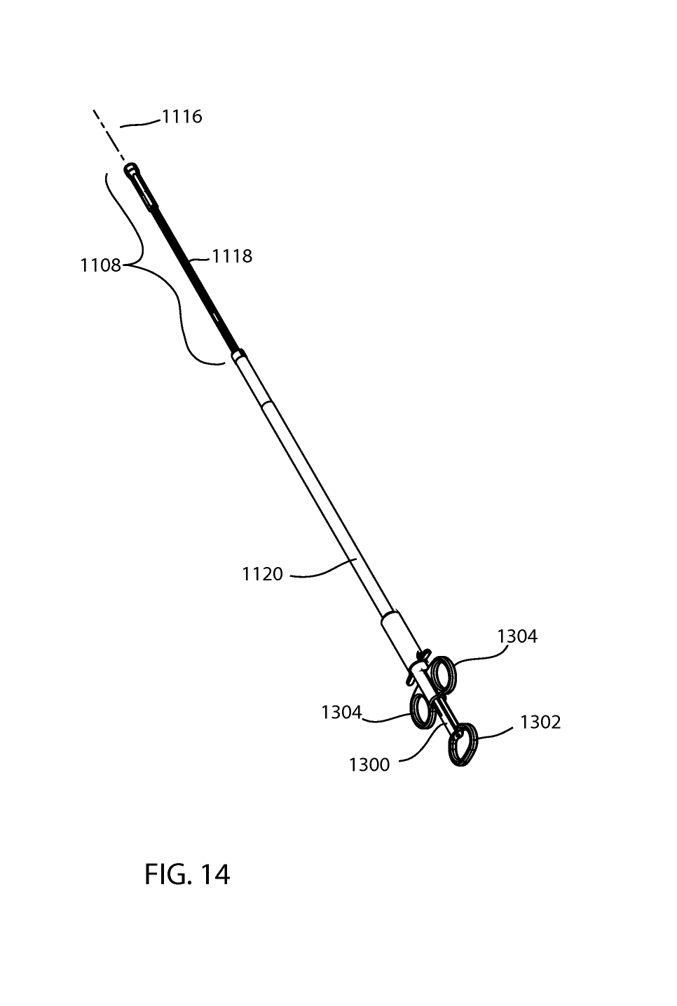

FIGS. 14-16 show how the surgical implant deployment member 1108 is deployed. In this example, the deployment actuation member 1122 includes a shaft 1300, thumb ring 1302, and finger rings 1304. The surgeon can use two fingers inserted into the finger rings 1304 and a thumb inserted into the thumb ring 1304 to actuate the surgical implant deployment device 1100. However, in other examples the deployment actuation member 1122 can include other structures to actuate the surgical implant deployment device 1100.

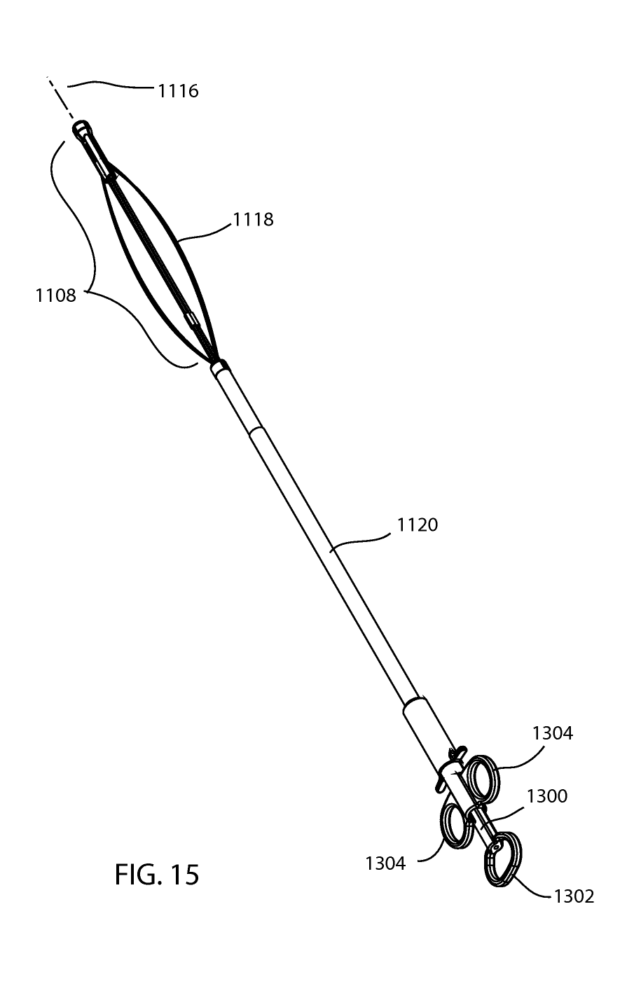

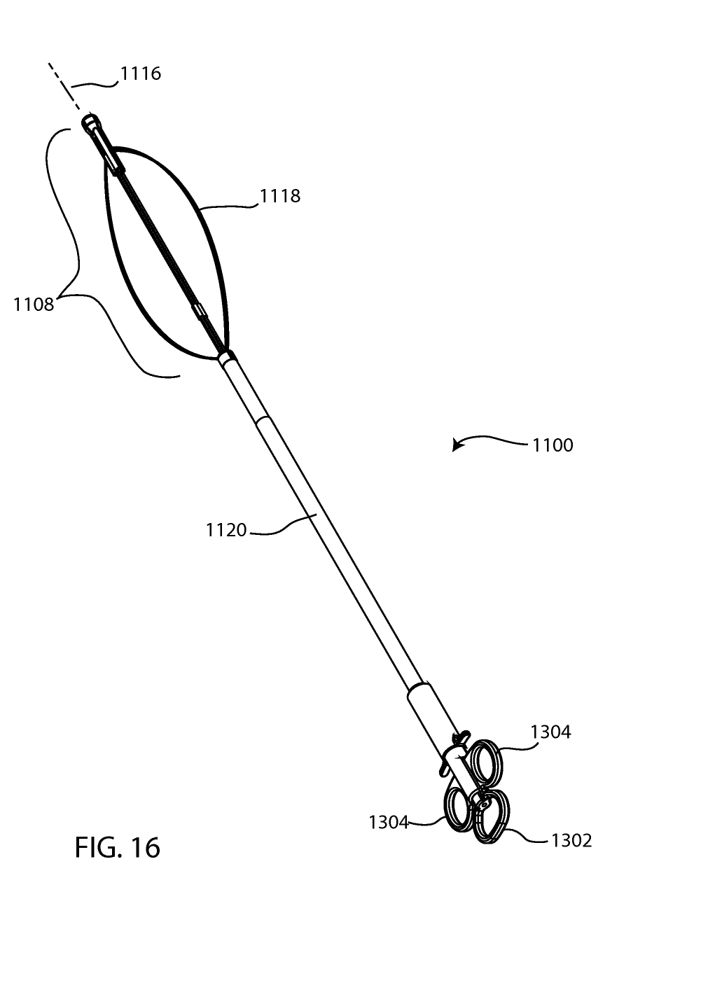

FIG. 14 shows the surgical implant deployment member 1108 in the retracted or “non-deployed” position with the shaft 1300 moved in the proximal direction away from the finger rings 1304. The elongate deploying members 1118 can be connected to the shaft 1300 through the elongate body 1120. Thus, as the thumb ring 1302 and shaft 1300 are pulled in the proximal direction, the elongate deploying members 1118 are also pulled in the proximal direction through the elongate body 1120, such that the elongate deploying members 1118 straighten out and approach the longitudinal axis 1116 of the surgical implant deployment member 108. FIG. 15 shows the surgical implant deployment member 1108 in a partially deployed configuration with the shaft 1300 partially moved in the distal direction. This moves the elongate deploying members 1118 in the distal direction and causes them to bend away from the longitudinal axis 1116 of the surgical implant deployment member 1108. FIG. 16 shows the surgical implant deployment device 1100 with the thumb ring 1302 and shaft 1300 fully depressed in the distal direction causing the elongate deploying members 1118 to deflect even further away from the longitudinal axis 1116 of the surgical implant deployment member 1108. This position is the “fully deployed” position. A surgical mesh 1102 trapped between the elongate retaining members 1112 and the elongate deployment members 1118 will be “unwrapped” or “unfurled” by the elongate deployment members 1118 as the elongate deployment members 1118 deflect away from the longitudinal axis 1116.

The elongate deploying members 1118 in this example are configured to deflect away from the longitudinal axis 1116 of the surgical implant deployment member 1108 in substantially the same plane. In other examples, the elongate deploying members 1118 can be configured to deflect away from the longitudinal axis 1116 of the surgical implant deployment member 1108 in more than one plane or along a curved surface. The distal end of the elongate body 1120 can have slits 1450 (see FIG. 9) configured to receive the elongate deploying members 1118 as they deflect away from the longitudinal axis 1116. In some embodiments, the distal end of the elongate body 1120 can have a cap member 1452 (see FIG. 10) with slits 1450 formed therein o for receiving the elongate deploying members 1118 as they deflect away from the longitudinal

Once the surgical mesh 1102 is unfurled, the surgeon can tack or suture the surgical mesh 1102 in place around the edges of the surgical mesh 1120; retract the elongate deploying members 1118; release the tip members 1135, 1136 from each other; remove the deployment device 1100 from the patient; and perform additional tacking or suturing to the surgical mesh 1102 as needed.

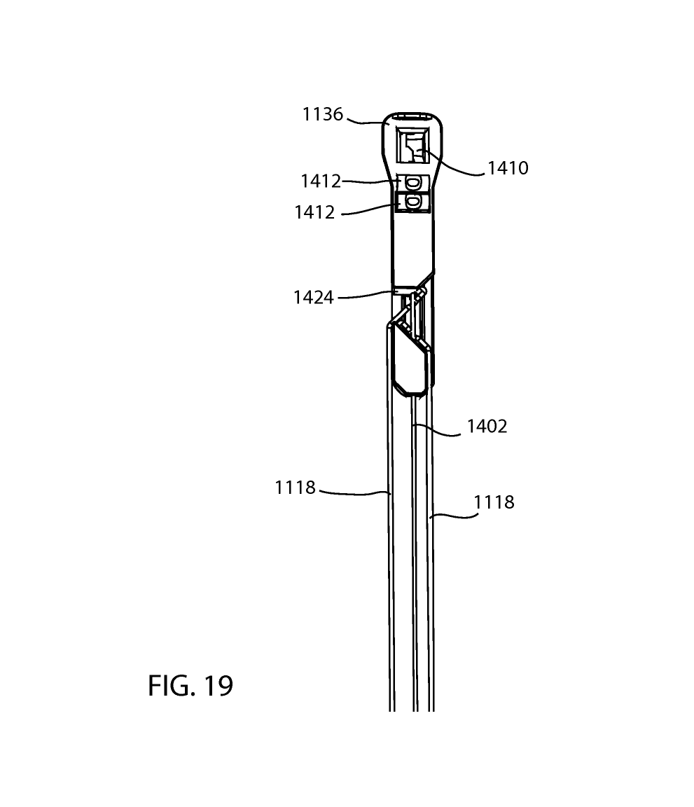

FIGS. 17-19 illustrate one example of an attachment mechanism 1404 configured to releasably attach the tip members 1135, 1136 to each other. The attachment mechanism 1404 can include a catch 1400 and a catch member 1402. The catch 1400 can be connected to the bottom tip member 1136. The catch 1400 can have a guide surface 1406 and a capture surface or hook 1408 below the guide surface 1406. The catch member 1402 can be a shaft that is axially moveable in the proximal-distal directions and connected to the top tip member 1135 (not shown in FIG. 17) through an aperture formed in the top tip member 1135. The catch member 1402 can be moved in the distal direction such that the catch member 1402 is exposed through the catch member window 1410 of the top tip member 1135 (see FIG. 19). The bottom tip member 1136 can then be urged toward the top tip member 1135 such that the catch 1400 enters the catch member window 1410 and the catch member or shaft 1402 is deflected sideways by the guide surface 1406 until the shaft 1402 is captured by hook 1408. The tip members 1135, 1136 are then attached to each other. Tip members 1135, 1136 can also have one or more bias members 1412 configured to impart a de-compressive force between the tip members 1135, 1136 to push the shaft 1402 against the hook 1408. The bias members 1412 can be made of a resilient material such as plastic or silicone.

The tip members 1135, 1136 can be released from each other by moving the catch member 1402 in the proximal direction away from the capture surface or hook 1408 of catch 1400. Once the catch member 1402 disengages from hook 1408, the de-compressive force from bias members 1412 push the tip members 1135, 1136 away from each other and the tip members 1135, 1136 separate.

In one example, the catch member or shaft 1402 can be attached to a release handle 1414 (see FIG. 8) through the elongate body 1120. The release handle 1414 can be releasably attached to the elongate body 1120 to prevent accidental release of the tip members 1135, 1136. The release handle 1414 can have tabs 1416 to facilitate the separation of the release handle 1414 from the elongate body 1120 by applying pressure to the tabs 1416. Once the release handle 1414 is separated from the elongate body 1120, the surgeon can pull the release handle in the proximal direction, causing the shaft 1402 to move in the proximal direction and releasing tip members 1135, 1136 from each other.

In an emergency, the elongate deploying members 1118 can be released from tip members 1135, 1136, or from the distal ends of the elongate retaining members 1112, via a release actuation member 1420. For example, if the surgeon accidentally places a tack or suture in the area between the elongate deploying members 1118 and the elongate retaining members 1112, the misplaced tack or suture can prevent the surgeon from subsequently retracting and removing the deployment device 1100 from the patient. In this emergency situation, the elongate deploying members 1118 can be released from tip members 1135, 1136 to allow the elongate deploying members 1118 to circumvent the misplaced tack and allow the deployment device 1100 to be removed from the patient.

FIG. 17 shows one example of a release actuation member 1420 for releasing the elongate deploying members 1118 from tip members 1135, 1136. In this example, the release actuation member 1420 shares some components with attachment mechanism 1404, specifically shaft 1402 and release handle 1414. As discussed previously, shaft 1402 can be moved proximal the catch 1400 to release the tip members 1135, 1136 from each other. However, if the surgeon continues to move shaft 1420 in the proximal direction, the shaft 1402 would eventually be proximal the bends 1422 formed in the elongate deployment members 1118, as seen in FIG. 17. Once the shaft 1402 is proximal the bends 1422, the elongate deployment members 1118 are then free to bend away from the top tip member 1135, as they are no longer constrained by shaft 1402. A similar release actuation member 1420 can be employed for the bottom tip member 1136 using a shaft 1428 to interact with bends formed in the elongate deploying members 1118 connected to the bottom tip member 1136.

In other examples, the elongate deployment members 1118 can have grooves (not shown) formed in the distal ends of the elongate deployment members 1118, instead of bends 1422, that are configured to interact with shafts 1402, 1428 to releasably attach the elongate deployment members 1118 to tip members 1135, 1136.

The tip members 1135, 1136 can have ramps or angled surfaces 1424 formed therein to help release the elongate deployment members 1118 from tip members 1135, 1136. The bends 1422 can translate in the proximal to distal direction along grooves 1426 formed in the tip members 1135, 1136. The bends 1422 can also rotate in the transverse direction in the grooves 1426 as the elongate deploying members 1118 deflect away from the longitudinal axis 1116.

The distal ends of the elongate deploying members 1118 can be made blunt to help prevent tissue injury as the elongate deploying members 1118 are released from the tip members 1135, 1136. The distal ends of the elongate deploying members 1118 can be dulled, rolled, bent or otherwise shaped to blunt the ends of the elongate deploying members 1118 to help prevent tissue injury as the elongate deploying members 1118 are released from the tip members 1135, 1136.

Any of the deployment devices 1100 described herein can include one or more guide members (not shown) configured to slide along the deployment device 1100 and change the shape of the elongate deploying members 1118 as they deflect away from the longitudinal axis 1116 of the surgical implant deployment member 1108. The guide member can have a body with one or more apertures formed therein for receiving the elongate retaining members 1112 and the elongate deploying members 1118 through the body. The guide member can then be forced in the distal to proximal direction along the elongate retaining members 1112 to change the shape of the elongate deploying members 1118 in the deployed configuration. For example, the guide member can be located on the proximal end of the surgical implant deployment member 1108 with the elongate deploying members 1118 in the deployed position. The guide member can then be forced in the distal direction, forcing the elongate deploying members 1118 to “thread” through the guide member and causing the deploying members 1118 to deflect further away from the longitudinal axis 1116.

Methods of using the deployment devices 1100 described herein can include: providing a surgical implant deployment device 1100 as described herein; loading a surgical implant 1102 onto the surgical implant deployment device 1100; inserting the surgical implant deployment device 1100, with the loaded surgical implant 1102 into a patient; moving the deployment actuation member 1122 in a first direction to deflect the elongate deploying members 1118 away from the longitudinal axis 1116 of the surgical implant deployment member 1108 and unfurl the surgical implant 1102; attaching the surgical implant 1102 to the patient; moving the deployment actuation member 1122 in a second direction causing the elongate deploying members 1118 to approach the longitudinal axis 1116 of the surgical implant deployment member 1108; releasing the distal ends of the elongate retaining members 1112 from each other; and removing the surgical implant deployment device 1100 from the patient.

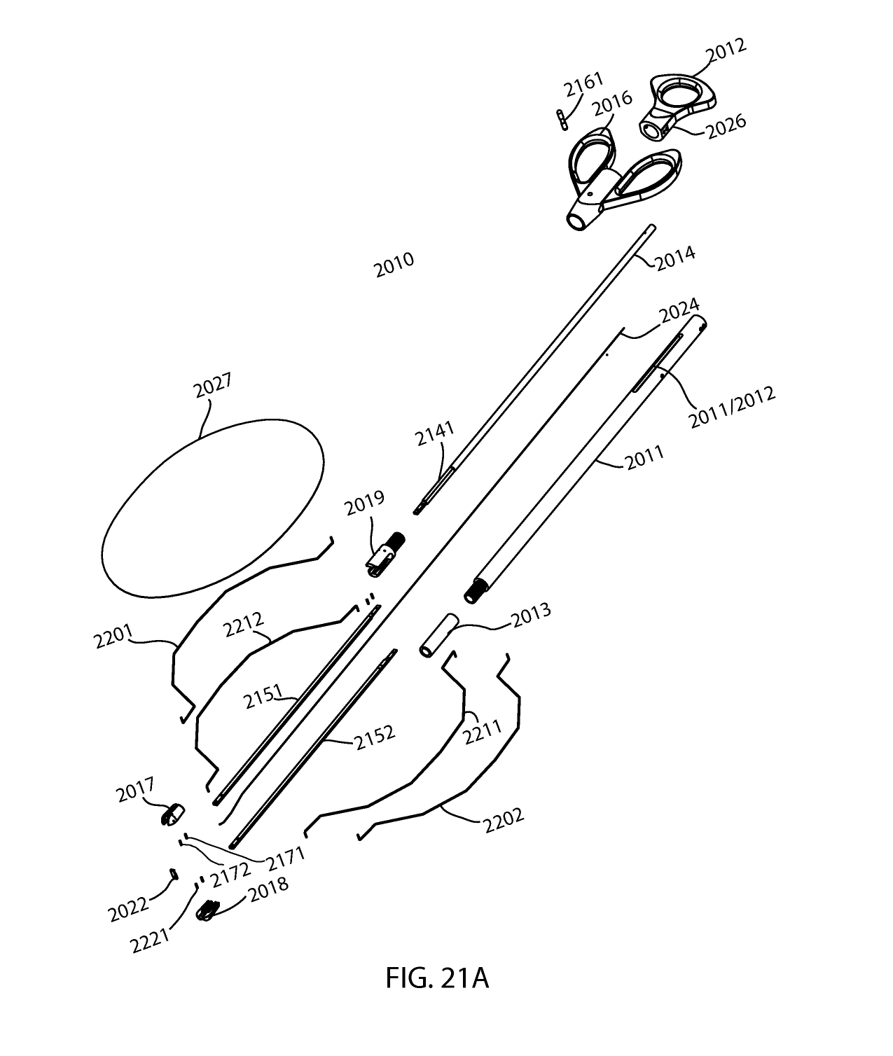

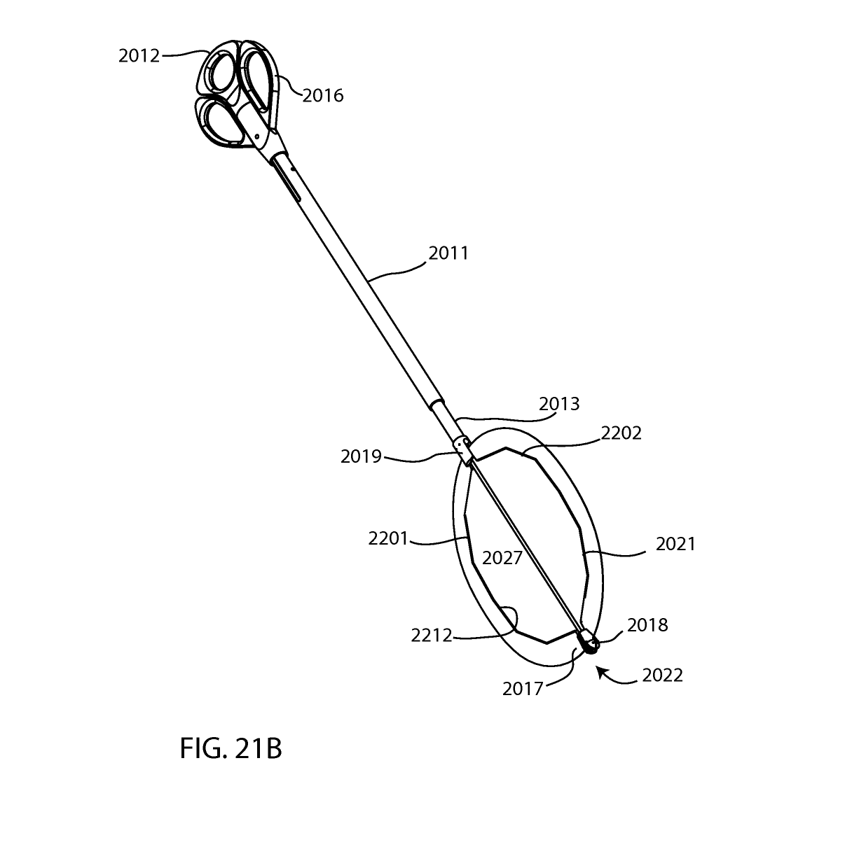

FIGS. 21A-25 illustrate a surgical implant deployment device 2010 according to another embodiment of the present disclosure. FIG. 21A is an exploded view of the mesh deployment apparatus 2010. The apparatus includes a main shaft 2011 having a fixed handle 2012 attached to one end. A hollow flexible tube 2013 can be attached to the other end of the main shaft 2011. The tube 2013 provides flexibility to the apparatus 2010 allowing for a greater range of motion to position and manipulate the mesh 2027. The main shaft 2011 is hollow and includes a moving handle securing slot 2111, and a latch release lever slot 2112, near the fixed handle 2012. The main shaft 2011 is preferably made from metal or biocompatible plastic.

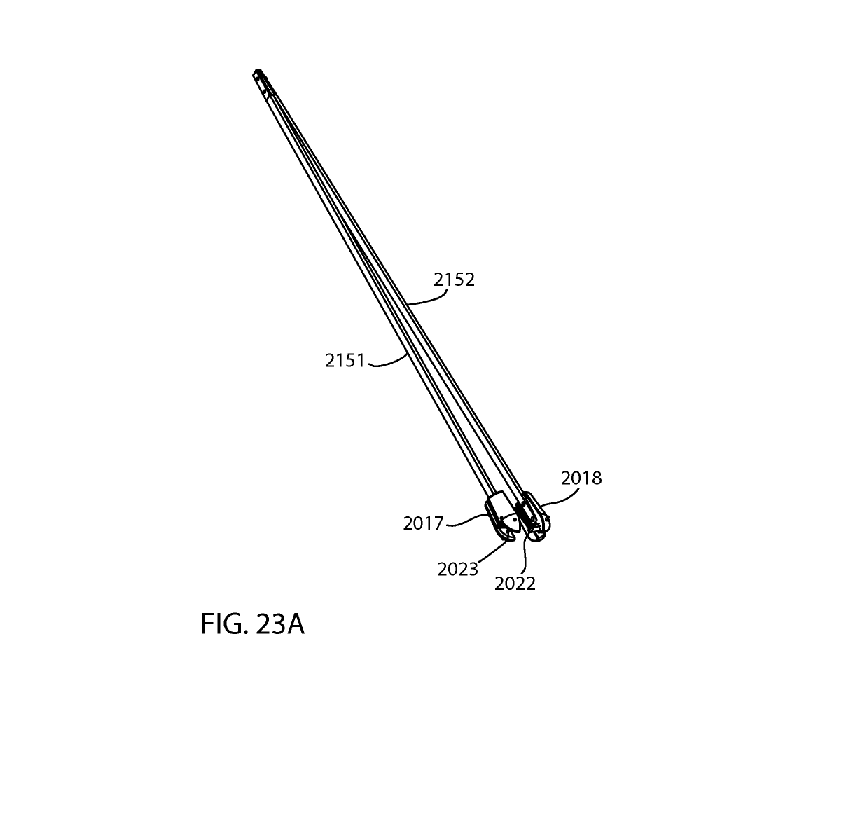

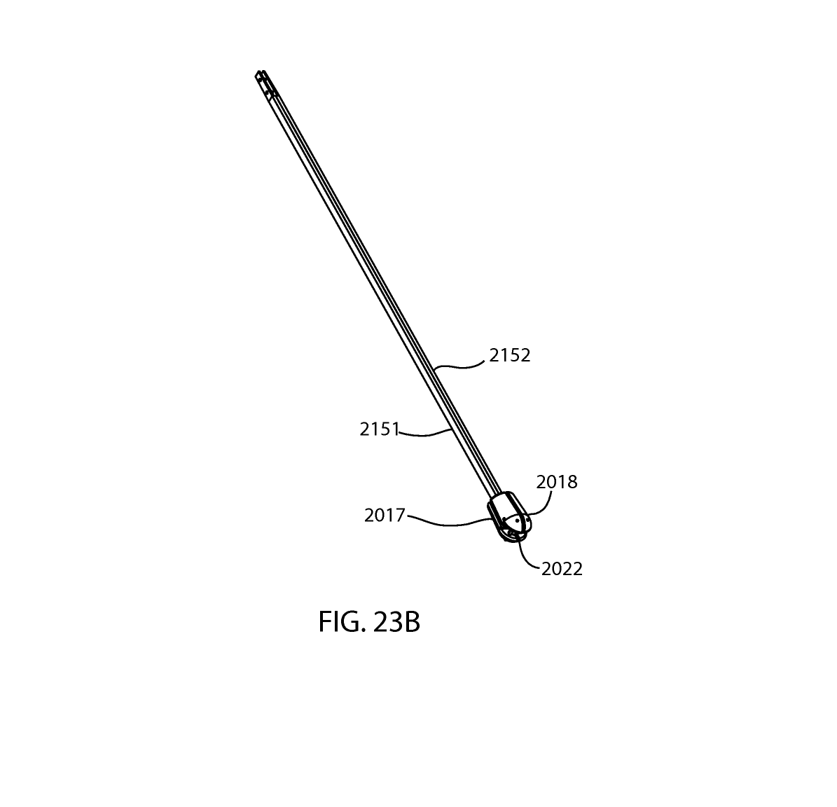

An actuator rod 2014 is connected to an actuator assembly 2015 at its distal end. The actuator rod preferably having a flexible portion 2141, at its distal end, said flexible portion preferably being made from a flexible plastic, such as Teflon PTFE, or rubber. The actuator rod 2014 being connected to a moving handle 2016 near its proximal end by a securing pin 2161, which passes through the main shaft via a securing slot 2111. An actuator assembly 2015, composed of two parallel bars, an upper parallel bar 2151 and a lower parallel bar 2152, connected to one another at their proximal ends, and two tip assemblies, an upper tip cap 2017, and a lower tip cap 2018, shown in detail in FIGS. 23A-23B and described in greater detail below, connected to a distal end of each parallel bar 2151, 2152 by connecting pins 2171, 2172, 2181, 2182. The actuator rod 2014, and actuator assembly 2015 are positioned internally to the main shaft 2011 and flexible tube 2013, such that the flexible portion 2141 of the actuator rod 2014 aligns with the flexible tube 2013 when the device is in the deployed position allowing the apparatus 2010 to flex for proper positioning of the mesh during use. Thus, the respective outer diameters of the actuator rod 2014, flexible portion 2141, and actuator assembly 2015 are smaller than the internal diameters of the hollow main shaft 2011 and flexible tube 2013.

A deployment arm carrier 2019 is affixed to an end of the flexible tube 2013. The deployment arm carrier 2019 includes an internal opening to allow the flexible portion of the actuator rod 2141 and proximal portion of the actuator assembly 2015 to slide through the deployment arm carrier 2019, and is preferably keyed to the flexible portion of the actuator rod 141 and proximal portion of the actuator assembly 2015 to prevent rotation about the long axis. An upper set of deployment arms 2201, 2202 and a lower set of deployment arms 2211, 2212, are attached to the deployment arm carrier 2019. The deployment arms 2201, 2202, 2211, 2212 are preferably pre-bent to maintain a semi-rigid shape and allow them to travel in a single fixed plane, and are preferably formed from spring steel, Nitinol, or similar elastic material which can return to its original shape after flexing, The upper set of deployment arms 2201, 2202, are preferably connected at their proximal end, symmetrically to the upper portion of carrier 2019, in the same plane, and on opposite sides of the carrier 2019, and connected at their distal end, symmetrically, to the upper tip cap 2017, in the same plane, and on opposite sides of the upper tip cap 2017. The lower set of deployment arms 2211, 2212, are preferably connected at their proximal end, symmetrically to the lower of carrier 2019, in the same plane, and on opposite sides of the carrier 2019, and connected at their distal end, symmetrically, to the lower tip cap 2017, in the same plane, and on opposite sides of the lower tip cap 2018. The connections fashioned in such a manner as to allow the deployment arms to move in only one plane, and such that the upper set of deployment arms 2201, 2202, and the lower set of deployment arms 2211, 2212 travel in the same or similar parallel planes.

The upper tip cap 2017 and a lower tip cap 2018 are preferably mounted on the distal ends of each of an upper 2151 and lower 2152 parallel bars by connecting pins 2171, 2172 and 2181, 2182. Each tip cap being functional to connect to the deployment arms 2201, 2202, 2211, 2212 as described above. The lower tip cap 2018 further housing a latch arm 2022 mounted to the lower tip cap 2018 by a latch pivot pin 2221, and the upper tip cap 2017 further housing a latch securing pin 2023. The latch arm 2022 being functional to engage the latch securing pin 2023, and operative to releasably secure the upper tip cap 2017 to the lower tip cap 2018 assuming a first closed position, and being functional to disengage the latch arm 2022 from the latch securing pin 2023 when pivoted about the pivot pin 2014 creating a second open position.

A latch release wire 2024 is connected at one end to the latch arm 2022, and at another end to a latch release lever 2025 connected to the moving handle 2016 via a latch release pivot pin 2251, the latch release wire 2024, running alongside the length of the actuator cap 2015 and actuator rod 2014, inside the deployment arm carrier 2019, flexible tube 2013, and main shaft 2011. The latch release lever 2025, connected to the latch arm 2022 via the latch release wire 2024, assumes a corresponding open position, in which the latch release lever 2025 is moved away from the direction of the latch arm 2022 when the latch arm 2022 is in its open position, and a corresponding closed position, in which the latch release lever 2025 is moved towards the direction of the latch arm 2022 when the latch arm 2022 is in its closed position. The latch arm 2022, latch release wire 2024, and latch release lever 2025 forming a latch release mechanism, having a first open, and a second closed configurations. A latch release lever activator 2026, mounted on, or being an integral part of, the fixed handle 2016, is operative to engage the latch release lever 2025 in its closed position, and is functional to pivot the latch release lever 2025 about the latch release pivot pin 2251, moving the latch release wire 2024 outward and causing the latch arm 2022 to pivot about the latch pivot pin 2221, causing the latch arm 2022 to disengage from the latch securing pin 2023, the latch mechanism assuming its open configuration, and thus disengaging the upper tip cap 2017 from the lower tip cap 2018.

Different variations of the above-described embodiment may be utilized. For example, for certain applications, the distal ends of the deployment arms and the latch mechanism may be connected to or fashioned from, the distal ends of the parallel bars without the use of tip assemblies. Furthermore, different materials may be substituted to construct the various components, as is known in the art.

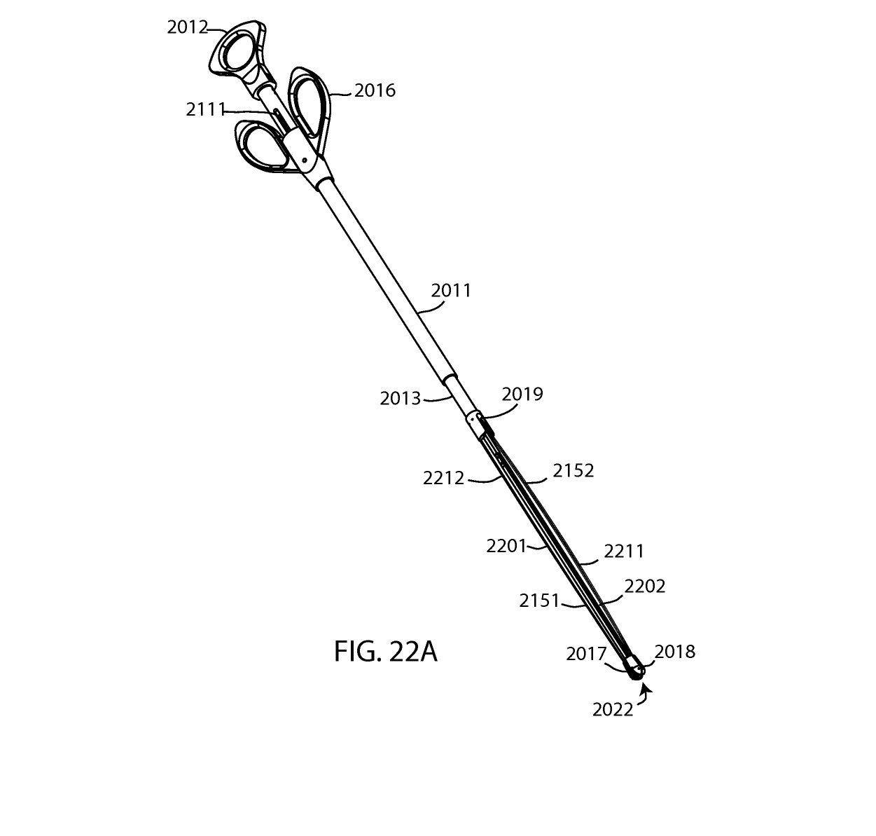

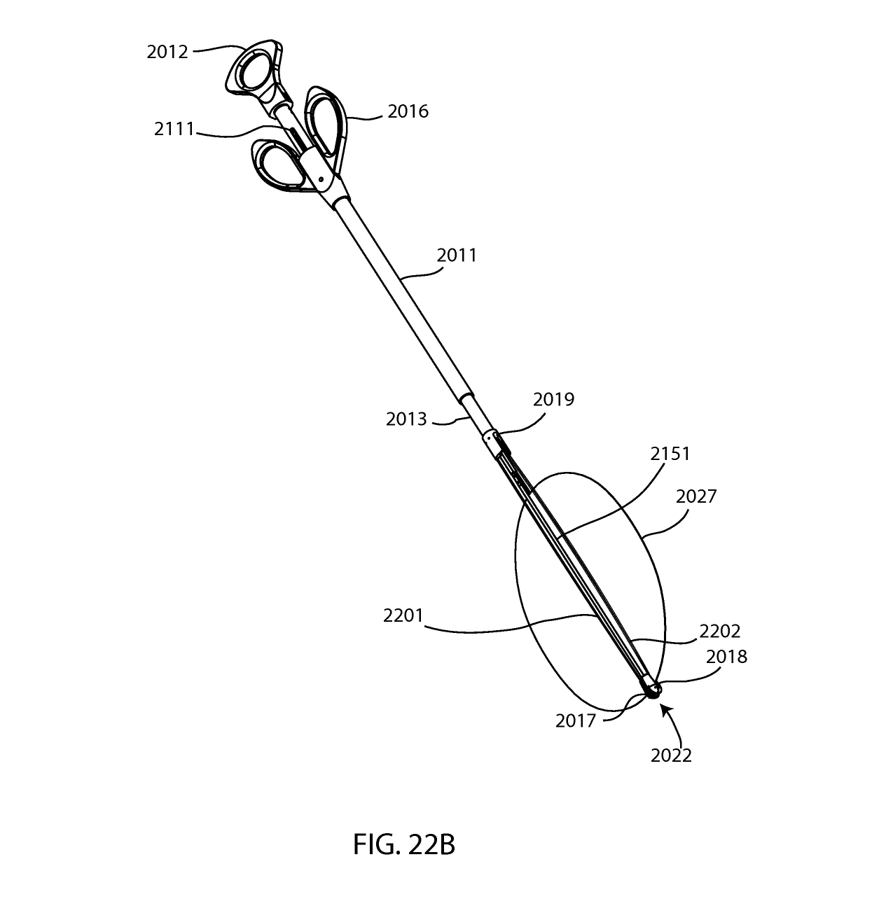

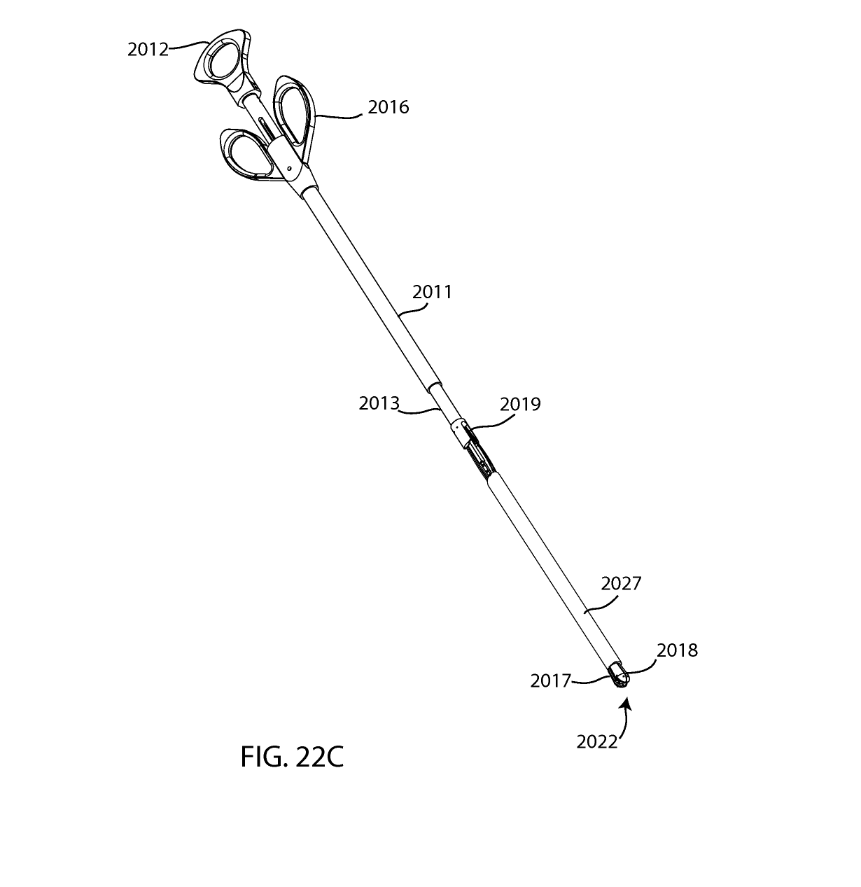

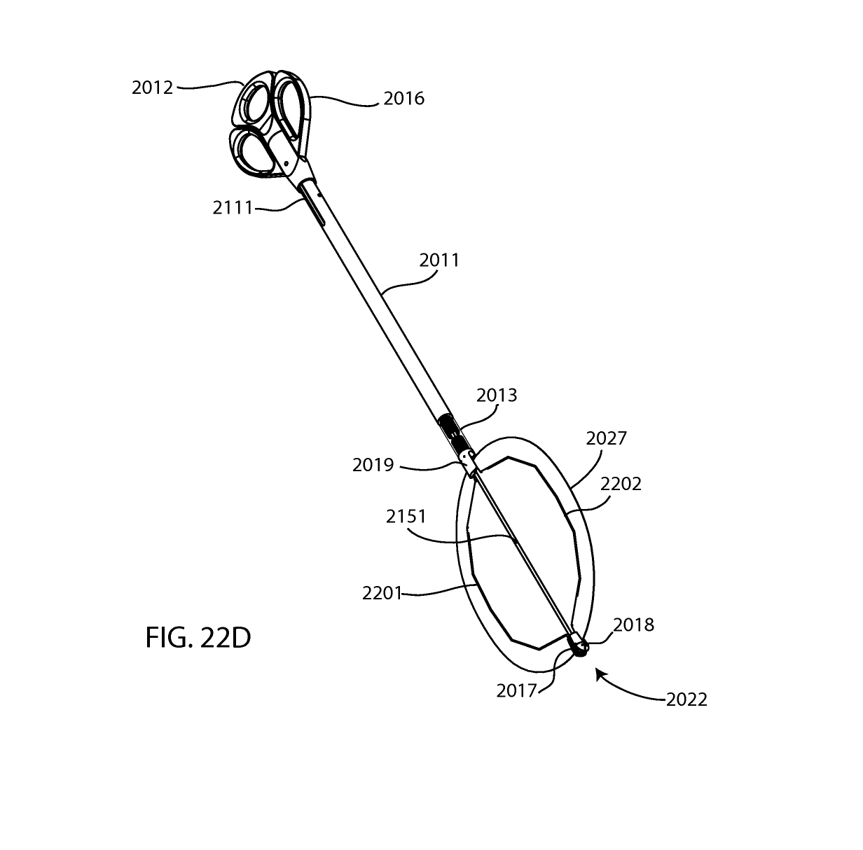

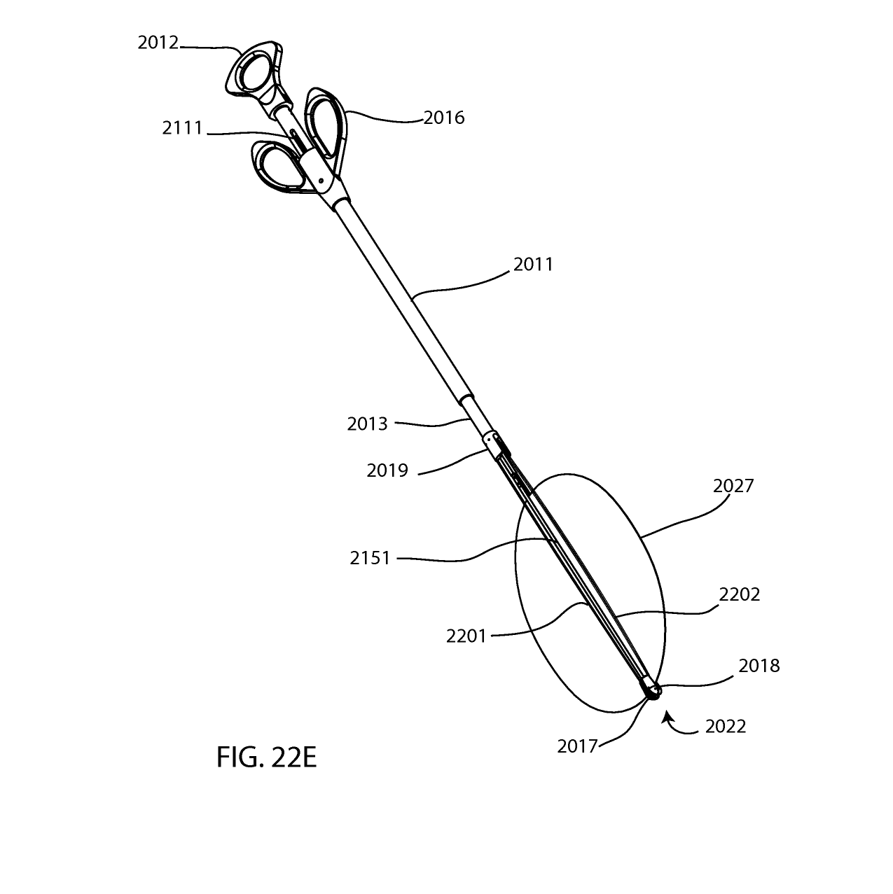

FIGS. 22A-22E illustrate the operation of the mesh deployment apparatus of FIG. 21A. The mesh deployment apparatus 2010 is in a first ready configuration with the fixed handle 2012 and movable handle 2016 extended away from one another. In this ready position (FIG. 22A) the two sets of deployment arms 2171, 2172, and 2181, 2182 are in a first retracted position. The latch mechanism is in its open configuration, with the latch arm 2022 disengaged from the latch securing pin 2023, and the latch release lever 2025, connected to the latch arm 2022 via the latch release wire 2024, in its corresponding open position. A surgical mesh 2027 is inserted between the upper parallel bar 2151 connected to the upper set of deployment arms 2201, 2202 via the upper tip cap 2017, and the lower parallel bar 2152 connected to the lower set of deployment arms 2211, 2212 via the lower tip cap 2018, as shown in FIG. 22B. The latch arm 2022 is then moved over the latch securing pin 2023, and the latch mechanism assumes its closed position, such that the mesh 2027 is firmly held between the upper 2151 and lower 2152 parallel bars and the mesh deployment apparatus assumes a closed ready configuration. The mesh 2027 is then rolled about the long axis of the actuator cap and deployment arms 2201, 2202, 2211, 2212 (FIG. 2C) causing the bends in the deployment arms 2201, 2202, 2211, 2212 to assume a loaded configuration. The device with mounted and rolled surgical mesh 2027 is then placed into a body cavity of a patient, preferably through a laparoscopic port (not shown). Once inside, the loaded deployment arms 2201, 2202, 2211, 2212 return first to the retracted position due to the stored energy in the bends of the deployment arms 2201, 2202, 2211, 2212, causing an initial un-furling of the mesh 2027 inside the body cavity of the patient. The two parts of the handle 2012, 2016 are then brought together causing the two sets of deployment arms 2201, 2202 and 2211, 2212 to expand into a second, deployed position, and causing the latch release lever activator 2026 in the fixed handle 2012 to engage the latch release lever 2025 in the moving handle 2016. The surgical mesh 2027, being trapped between the upper set of deployment arms 2201, 2202 and the lower set of deployment arms 2211, 2212, further unfurls into a flat, deployed configuration. The surgical mesh 2027 is then positioned over a hernia defect in a wall of the body cavity of the patient, positioning being aided by the flexible joint created by the flexible tube 2013 and the flexible portion of the actuator rod 2141. The mesh 2027 is then affixed to the wall of the body cavity by using any one of a standard surgical tacking or affixing device well known in the art. Once the mesh 2027 has been affixed to the wall of the body cavity, the two handles 2012, 2016 are moved away from one another, causing first, the latch release lever activator 2026 to pivot the latch release lever 2025 to its open position, and causing the latch mechanism to assume an open configuration, with the latch arm 2022 becoming disengaged from the latch securing pin 2023. Further movement of the handles 2012, 2016 away from one another causes the deployment arms 2201, 2201, 2211, 2212 to return to their retracted position, and the device to return to its ready configuration. In this configuration, the mesh 2027 is no longer firmly held between the upper 2151 and lower 2152 parallel bars and the device 2010 can be safely removed from the body cavity of the patient.

FIGS. 23A-23B are perspective detailed views of an actuator assembly 2015. FIG. 23A shows the latch arm 2022 in the open position. The latch arm 2022, housed in the lower tip cap 2018, is disengaged from the latch securing pin 2023, housed in the upper tip cap 2017, and the latch release wire 2024 is in its open position. In this position, the two parallel bars 2151, 2152 are disengaged from one another. FIG. 23B shows the latch arm 2022 in the closed position. The latch arm 2022 is engaged with the latch securing pin 2023 such that the two tip caps 2022, 2023 connected to the two parallel bars 2151, 2152 are engaged to one another.

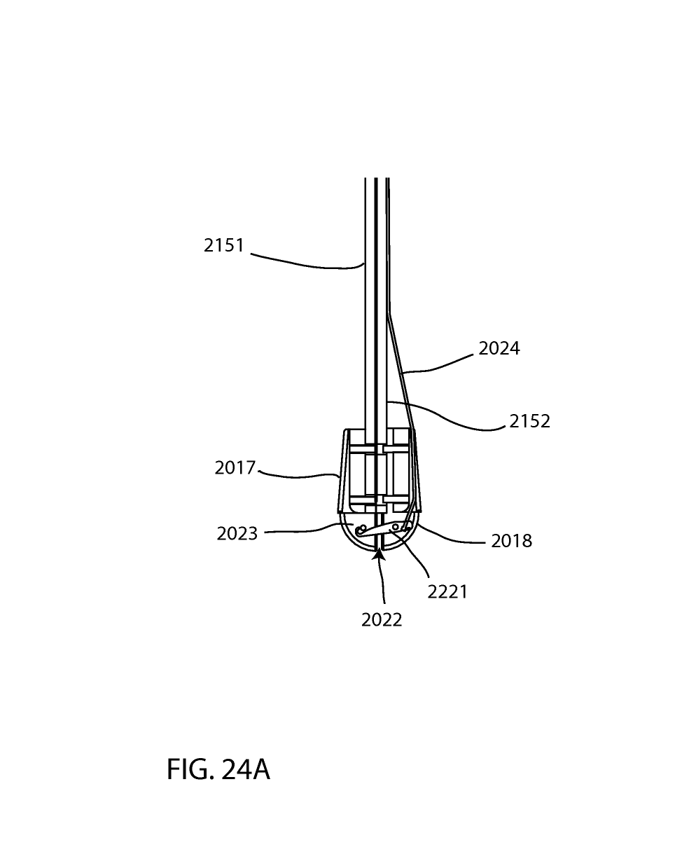

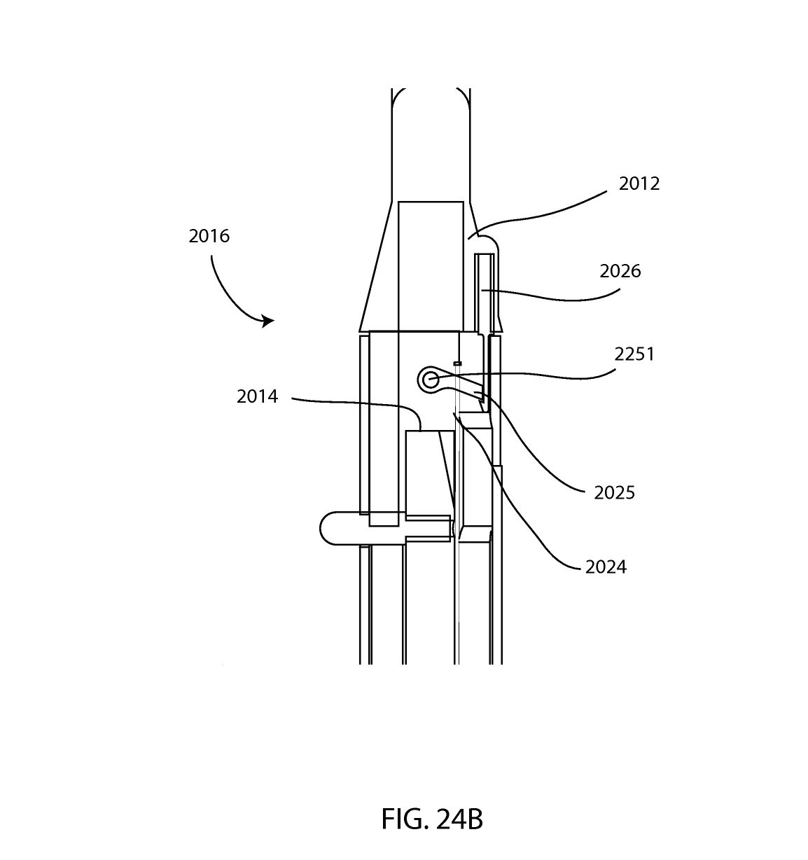

FIG. 24A is a perspective view of the latch release mechanism in the closed position. The latch release lever activator 2026 is engaged with the latch release lever 2025 and the latch release wire 2024 is in its closed position. Movement of the fixed handle 2012 and moving handle 2016 away from one another causes the latch release lever activator 2026 to pivot the latch release lever 2025 about the latch release pivot pin 2251, pulling the latch release wire 2024 outward, functional to disengage the latch arm 2022 from the latch securing pin 2023. Once the latch release lever activator 2026 has pivoted the latch release lever 2025 to its open position, it becomes disengaged from the latch release lever 2025, allowing the fixed 2012 and moving 2016 handles to be moved away from one another into their retracted position.

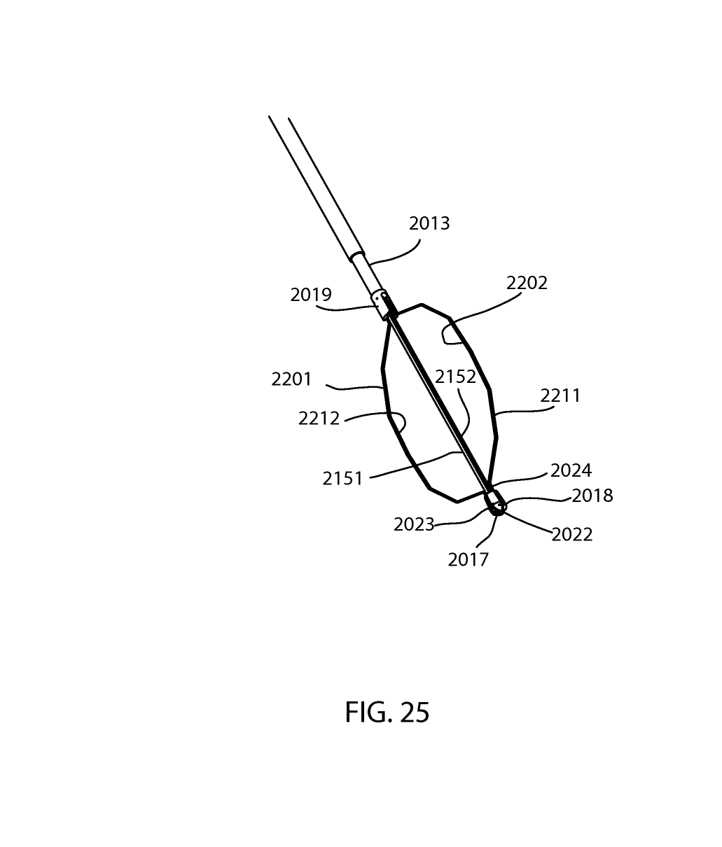

FIG. 25 is a perspective detailed view of the distal portion of the mesh deployment apparatus 2010 of FIG. 21A with the deployment arms 2201, 2202, 2211, 2212 in their deployed position and the latch mechanism in its closed configuration. The upper set of deployment arms 2201, 2202 are connected at their proximal ends to the upper portion of the deployment arm carrier 2019 and at their distal ends to the upper tip cap. The lower set of deployment arms 2211, 2212 are connected at their proximal ends to the lower portion of the deployment arm carrier 2019 and at their distal ends to the lower tip cap, the connections being fashioned such that the upper and lower sets of deployment arms 2201, 2202, 2211, 2212 expand outward to, and retract inward from, their deployed position in fixed planes which are essentially parallel to one another.

Those skilled in the art will appreciate that various adaptations and modifications of the just described preferred embodiments can be configured without departing from the scope and spirit of the invention. Therefore, it is to be understood that, within the scope of the above description, the invention may be practiced other than as specifically described herein.

It should be understood that the present apparatuses and methods are not intended to be limited to the particular forms disclosed. Rather, they are intended to include all modifications, equivalents, and alternatives falling within the scope of the claims. They are further intended to include embodiments which may be formed by combining features from the disclosed embodiments, and variants thereof.

The claims are not to be interpreted as including means-plus- or step-plus-function limitations, unless such a limitation is explicitly recited in a given claim using the phrase(s) “means for” or “step for,” respectively.

The term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

The present invention may be embodied in other specific forms without departing from its spirit or essential characteristics. It is appreciated that various features of the above-described examples can be mixed and matched to form a variety of other alternatives. For example, attachment members, deploying arms, etc., may be interchangeable in any of the embodiments set forth herein. As such, the described embodiments are to be considered in all respects only as illustrative and not restrictive. Similarly, manufacturing, assembly methods, and materials described for one device may be used in the manufacture or assembly of another device. The scope of the invention is, therefore, indicated by the appended claims rather than by the foregoing description. All changes which come within the meaning and range of equivalency of the claims are to be embraced within their scope.

1. A surgical implant deployment device comprising:
an elongate body having a proximal end and a distal end;
a deployment actuation member disposed at the proximal end of the elongate body; and
a surgical implant deployment member disposed at the distal end of the elongate body, the surgical implant deployment member comprising:
a longitudinal axis;
at least two elongate retaining members arranged opposite each other along the longitudinal axis, each of the at least two elongate retaining members having a proximal end and a distal end, wherein the proximal ends of the at least two elongate retaining members are adjacent the elongate body and the distal ends of the at least two elongate retaining members are distal from the elongate body, wherein the at least two elongate retaining members are configured to impart a compressive force between each other; and
at least two elongate deploying members arranged opposite each other along a side of the at least two elongate retaining members, each of the at least two elongate deploying members having a proximal end and a distal end, wherein the proximal ends of the at least two elongate deploying members are adjacent the elongate body and the distal ends of the at least two elongate deploying members are distal from the elongate body, and wherein the distal end of one of the at least two elongate deploying members is engaged with the distal end of one of the at least two elongate retaining members and the distal end of the other of the at least two elongate deploying members is engaged with the distal end of the other of the at least two elongate retaining members, the surgical implant deployment member configured to receive a surgical implant between the at least two elongate retaining members and the at least two elongate deploying members;
wherein moving the deployment actuation member in a first direction causes the at least two elongate deploying members to deflect away from the longitudinal axis of the surgical implant deployment member in substantially the same plane, and wherein moving the deployment actuation member in a second direction causes the at least two elongate deploying members to approach the longitudinal axis of the surgical implant deployment member.

2. The surgical implant deployment device of claim 1, further comprising a compression release member configured to decompress the compressive force between the at least two elongate retaining members.

3. The surgical implant deployment device of claim 1, further comprising a release actuation member configured to releasably attach the distal ends of the at least two elongate deploying members from the distal ends of the at least two elongate retaining members.

4. The surgical implant deployment device of claim 3, wherein the release actuation member comprises a release handle coupled to at least one release shaft, wherein when the at least one release shaft is moved to a first position, the distal ends of the at least two elongate deploying members are attached to the distal ends of the at least two elongate retaining members, and wherein when the at least one release shaft is moved to a second position, the distal ends of the at least two elongate deploying members are released from the distal ends of the at least two elongate retaining members.

5. The surgical implant deployment device of claim 4 comprising two release shafts.

6. The surgical implant deployment device of claim 4 further comprising bends formed in the distal ends of the at least two elongate deploying members and configured to interact with the at least one release shaft to releasably attach the distal ends of the at least two elongate deploying members to the distal ends of the at least two elongate retaining members.

7. The surgical implant deployment device of claim 4 further comprising grooves formed in the distal ends of the at least two elongate retaining members configured to interact with the at least one release shaft to releasably attach the distal ends of the at least two elongate deploying members to the distal ends of the at least two elongate retaining members.

8. The surgical implant deployment device of claim 1 wherein the distal ends of the at least two elongate deploying members are blunt.

9. The surgical implant deployment device of claim 1 further comprising an attachment mechanism configured to releasably attach the distal ends of the at least two elongate retaining members to each other.

10. The surgical implant deployment device of claim 9, wherein the attachment mechanism comprises a catch associated with one of the at least two elongate retaining members configured to interact with a capture member associated with the other of the at least two elongate retaining members.

11. The surgical implant deployment device of claim 10, wherein the catch is a hook and the capture member is a shaft, wherein when the shaft is moved to a first position, the hook engages the shaft to attach the distal ends of the at least two elongate retaining members to each other, and wherein when the shaft is moved to a second position, the hook disengages the shaft and releases the distal ends of the at least two elongate retaining members from each other.

12. The surgical implant deployment device of claim 1, further comprising at least one grip member disposed along the at least two elongate retaining members and configured to grip a surgical implant disposed between the at least two elongate retaining members.

13. The surgical implant deployment device of claim 12, comprising four grip members disposed along the at least two elongate retaining members.

14. The surgical implant deployment device of claim 12, wherein the length of the at least one grip member is adjustable.

15. The surgical implant deployment device of claim 12, wherein the at least one grip member comprises polyethylene foam.

16. The surgical implant deployment device of claim 12, wherein the at least one grip member comprises silicone.

17. The surgical implant deployment device of claim 12, wherein the at least one grip member comprises indentations formed in the at least two elongate retaining members and configured to grip a surgical implant disposed between the at least two elongate retaining members.

18. The surgical implant deployment device of claim 1 further comprising a flexible joint intermediate the surgical implant deployment member and the elongate body.

19. The surgical implant deployment device of claim 18 wherein the flexible joint comprises a length of flexible plastic.

20. The surgical implant deployment device of claim 1, wherein the distal ends of the at least two elongate retaining members form an implant shield having a diameter greater than or equal to the diameter of a surgical implant loaded onto the surgical implant deployment device in a pre-deployment configuration.

21. The surgical implant deployment device of claim 20, wherein the diameter of the implant shield is about 12 mm.

22. The surgical implant deployment device of claim 1, further comprising a guide member configured to slide along the surgical implant deployment member and change the shape of the elongate deploying members as they deflect away from the longitudinal axis of the surgical implant deployment member.

23. A surgical implant deployment device comprising:
an elongate body having a proximal end and a distal end;
a deployment actuation member disposed at the proximal end of the elongate body; and
a surgical implant deployment member disposed at the distal end of the elongate body, the surgical implant deployment member comprising:
a longitudinal axis;
two elongate retaining members arranged opposite each other along the longitudinal axis, each of the two elongate retaining members having a proximal end and a distal end, wherein the proximal ends of the two elongate retaining members are adjacent the elongate body and the distal ends of the two elongate retaining members are distal from the elongate body, wherein the two elongate retaining members are configured to impart a compressive force between each other;
a first pair of elongate deploying members arranged opposite each other along a first side of the two elongate retaining members; and
a second pair of elongate deploying members arranged opposite each other along a second side of the two elongate retaining members;
each of the elongate deploying members having a proximal end and a distal end, wherein the proximal ends of the elongate deploying members are adjacent the elongate body and the distal ends of the elongate deploying members are distal from the elongate body;
wherein the distal end of one of the first pair of elongate deploying members and the distal end of one of the second pair of elongate deploying members are engaged with the distal end of one of the two elongate retaining members and the distal end of the other of the first pair of elongate deploying members and the distal end of the other of the second pair of elongate deploying members are engaged with the distal end of the other of the two elongate retaining members;
wherein moving the deployment actuation member in a first direction causes the first and second pairs of elongate deploying members to deflect away from the longitudinal axis of the surgical implant deployment member in substantially the same plane, and wherein moving the deployment actuation member in a second direction causes the first and second pairs of elongate deploying members to approach the longitudinal axis of the surgical implant deployment member.

24. The surgical implant deployment device of claim 23, further comprising a compression release member configured to decompress the compressive force between the two elongate retaining members.

25. The surgical implant deployment device of claim 24, further comprising a release actuation member configured to releasably attach the distal ends of the first and second pairs of elongate deploying members from the distal ends of the two elongate retaining members.

26. The surgical implant deployment device of claim 24, wherein the release actuation member comprises a release handle coupled to two release shafts, wherein when the two release shafts are moved to a first position, the distal ends of the first and second pairs of elongate deploying members are attached to the distal ends of the two elongate retaining members, and wherein when the two release shafts are moved to a second position, the distal ends of the first and second pairs of elongate deploying members are released from the distal ends of the two elongate retaining members.

27. The surgical implant deployment device of claim 26 wherein the release handle is removably coupled to the elongate body.

28. The surgical implant deployment device of claim 26 further comprising bends formed in the distal ends of the first and second pairs of elongate deploying members and configured to interact with the two release shafts to releasably attach the distal ends of the first and second pairs of elongate deploying members to the distal ends of the two elongate retaining members.

29. The surgical implant deployment device of claim 26 further comprising grooves formed in the distal ends of the first and second pairs of elongate deploying members and configured to interact with the two release shafts to releasably attach the distal ends of the first and second pairs of elongate deploying members to the distal ends of the two elongate retaining members.

30. The surgical implant deployment device of claim 23 wherein the distal ends of the first and second pairs of elongate deploying members are blunt.

31. The surgical implant deployment device of claim 23 further comprising an attachment mechanism configured to releasably attach the distal ends of the two elongate retaining members to each other.

32. The surgical implant deployment device of claim 31, wherein the attachment mechanism comprises a hook associated with one of the two elongate retaining members and configured to interact with a capture member associated with the other of the two elongate retaining members.

33. The surgical implant deployment device of claim 32, wherein the capture member is a shaft, wherein when the shaft is moved to a first position the hook engages the shaft to attach the distal ends of the at two elongate retaining members to each other, and wherein when the shaft is moved to a second position the hook disengages the shaft and releases the distal ends of the two elongate retaining members from each other.

34. The surgical implant deployment device of claim 23, further comprising at least one grip member disposed along the two elongate retaining members and configured to grip a surgical implant disposed between the two elongate retaining members.

35. The surgical implant deployment device of claim 34, comprising four grip members disposed along the two elongate retaining members.

36. The surgical implant deployment device of claim 34, wherein the length of the at least one grip member is adjustable.

37. The surgical implant deployment device of claim 34, wherein the at least one grip member comprises polyethylene foam.

38. The surgical implant deployment device of claim 34, wherein the at least one grip member comprises silicone.

39. The surgical implant deployment device of claim 34, wherein the at least one grip member comprises indentations formed in at least one of the two elongate retaining members and configured to grip a surgical implant disposed between the two elongate retaining members.

40. The surgical implant deployment device of claim 23 further comprising a flexible joint intermediate the surgical implant deployment member and the elongate body.

41. The surgical implant deployment device of claim 40 wherein the flexible joint comprises a length of flexible plastic.

42. The surgical implant deployment device of claim 23, wherein the distal ends of the two elongate retaining members form an implant shield having a diameter greater than or equal to the diameter of a surgical implant in the loaded configuration.

43. The surgical implant deployment device of claim 42, wherein the diameter of the implant shield is about 12 mm.

44. The surgical implant deployment device of claim 23, further comprising a guide member configured to slide along the surgical implant deployment member and change the shape of the first and second pairs of elongate deploying members as they deflect away from the longitudinal axis of the surgical implant deployment member.

45. A method of deploying a surgical implant comprising:
providing a surgical implant deployment device comprising:
an elongate body having a proximal end and a distal end;
a deployment actuation member disposed at the proximal end of the elongate body; and
a surgical implant deployment member disposed at the distal end of the elongate body, the surgical implant deployment member comprising:
a longitudinal axis;
two elongate retaining members arranged opposite each other along the longitudinal axis, each of the two elongate retaining members having a proximal end and a distal end, wherein the proximal ends of the two elongate retaining members are adjacent the elongate body and the distal ends of the two elongate retaining members are distal from the elongate body, wherein the two elongate retaining members are configured to impart a compressive force between each other;
a first pair of elongate deploying members arranged opposite each other along a first side of the two elongate retaining members; and
a second pair of elongate deploying members arranged opposite each other along a second side of the two elongate retaining members;
each of the elongate deploying members having a proximal end and a distal end, wherein the proximal ends of the elongate deploying members are adjacent the elongate body and the distal ends of the elongate deploying members are distal from the elongate body;
wherein the distal end of one of the first pair of elongate deploying members and the distal end of one of the second pair of elongate deploying members are engaged with the distal end of one of the two elongate retaining members and the distal end of the other of the first pair of elongate deploying members and the distal end of the other of the second pair of elongate deploying members are engaged with the distal end of the other of the two elongate retaining members;
wherein moving the deployment actuation member in a first direction causes the first and second pairs of elongate deploying members to deflect away from the longitudinal axis of the surgical implant deployment member in substantially the same plane, and wherein moving the deployment actuation member in a second direction causes the first and second pairs of elongate deploying members to approach the longitudinal axis of the surgical implant deployment member;
loading a surgical implant onto the surgical implant deployment device;
inserting the surgical implant deployment device with the loaded surgical implant into a patient; and
moving the deployment actuation member in a first direction to deflect the first and second pairs of elongate deploying members away from the longitudinal axis of the surgical implant deployment member and unfurling the surgical implant.

46. The method of claim 45 further comprising:
moving the deployment actuation member in a second direction causing the first and second pairs of elongate deploying members to approach the longitudinal axis of the surgical implant deployment member;
releasing the distal ends of the two elongate retaining members from each other; and
removing the surgical implant deployment device from the patient.
